SEQUENCE LISTING (15 sequences)

SEQ ID NO: 1 - DNA, 678 bp, Artificial
  Vacuolar H ATPase subunit E (vha-8) perfect gene
atgggcatttccgacaacgatgtccaaaaacagcttcgccatatgatggcatttattgagcaggaagcgaacgaaaaagcagaagaaatcgacgcaaaagcggaggaggagtttaacattgagaagggaaaattggtgcaactgcaaaggcagaaaatcatggaatattacgagaagaaagagaagcaagtggagctgcagcgcaagattcagaggtcgaatatgcaaaatcagagtcggttgaaatgtttgaaggcgcgggacgaccatttgaaaaatgtgttggaggaggcacgcgccaatctcagcaaaatttcggcggacagagagcgctacccggccattctcaaaggacttttgcttcagggccttttccaattgctcgaaagcaaagttgtgttgcgatgccgcaaaaaagacgaggaaatggtggcgaggattttgcccgaatgtttggaggaagttcagcggacgtggggcaaccgaagcgaagtgaaaattgacaatgagcactttttgtccccggaaagtgcgggcggcgtcgaattgttggccaaagacggcaaaatccgcgtctcgtccactttggaagcgcgtctcgacctcattgccgacaaaatcactccccaagttcgcactgctctgttcggtcccaatcagaaccgccgcttcttcgac

SEQ ID NO: 2 - DNA, 609 bp, Artificial
  Elongation factor 1 alpha (eft-4) perfect gene
gcgattcgcaaggaaaagatccatatcaatattgtggtgatcggtcacgtcgattccggcaaatcgactaccactggccatctcatctacaaatgtggtggaattgacaaacgtaccatcgagaaattcgagaaggaggctcaggaaatgggcaagggatcgtttaaatacgcttgggtgcttgacaagctgaaagccgaacgcgagcgtggtatcactatcgacatcgctttgtggaaattcgagacaggcaagtactatgtcaccattattgacgctcctggtcatcgagacttcgtcaaaaacatgatcacagggacttctcaagctgactgcgctgtgctggtggttgcttgtggaacgggagagtttgaggctggaatttcgaaaaacggccaaacgcgtgagcacgctttgcttgcgcagactttgggggtcaagcagctcatcgttgcttgcaacaagatggacaccactgaaccgccattcgccgaggcccgttatacggaagtgatgactgaagtgtccaacttcatcaagaagaccggttacaacccagccaccgttccattcgtcccaatctctggcttcaatggcgacaacatgtta

SEQ ID NO: 3 - DNA, 609 bp, Artificial
  Mismatch Elongation factor 1 alpha (eft-4)
gcgattcgcaaggaaaagaaccatatcaatattgtggtgttcggtcacgtcgattccgggaaatcgactaccactggccttctcatctacaaatgtggtcgaattgacaaacgtaccatggagaaattcgagaaggagggtcaggaaatgggcaagggaacgtttaaatacgcttgggtccttgacaagctgaaagccgtacgcgagcgtggtatcactttcgacatcgctttgtggaatttcgagacaggcaagtactttgtcaccattattgacgctgctggtcatcgagacttcgtgaaaaacatgatcacagggagttctcaagctgactgcgctctgctggtggttgcttgtggtacgggagagtttgaggctgcaatttcgaaaaacggccaatcgcgtgagcacgctttgctagcgcagactttgggggtcatgcagctcatcgttgcttgctacaagatggacaccactgatccgccattcgccgaggccccttatacggaagtgatgactcaagtgtccaacttcatcaacaagaccggttacaacccaggcaccgttccattcgtcccattctctggcttcaatggcgagaacatgtta

SEQ ID NO: 4 - DNA, 20 bp, Artificial
  Hsvha-8 forward primer
atgggcatttccgacaacga

SEQ ID NO: 5 - DNA, 20 bp, Artificial
  Hsvha-8 reverse primer
atgggcatttccgacaacga

SEQ ID NO: 6 - DNA, 29 bp, Artificial
  Hsvha-8 forward sense cDNA primer
taactcgagatgggcatttccgacaacga

SEQ ID NO: 7 - DNA, 29 bp, Artificial
  Hsvha-8 reverse sense cDNA primer
taaggtacctgattttgtcggcaatgagg

SEQ ID NO: 8 - DNA, 29 bp, Artificial
  Hsvha-8 forward antisense cDNA primer
taatctagaatgggcatttccgacaacga

SEQ ID NO: 9 - DNA, 29 bp, Artificial
  Hsvha-8 reverse antisense cDNA primer
taaatcgattgattttgtcggcaatgagg

SEQ ID NO: 10 - DNA, 19 bp, Artificial
  HsEf1a forward primer
gcgattcgcaaggaaaaga

SEQ ID NO: 11 - DNA, 20 bp, Artificial
  HsEf1a reverse primer
taacatgttgtcgccattga

SEQ ID NO: 12 - DNA, 28 bp, Artificial
  HsEf1a forward sense cDNA primer
taactcgaggcgattcgcaaggaaaaga

SEQ ID NO: 13 - DNA, 29 bp, Artificial
  HsEf1a reverse sense cDNA primer
taaggtacctaacatgttgtcgccattga

SEQ ID NO: 14 - DNA, 28 bp, Artificial
  HsEf1a forward antisense cDNA primer
taatctagagcgattcgcaaggaaaaga

SEQ ID NO: 15 - DNA, 29 bp, Artificial
  HsEf1a reverse antisense cDNA primer
taaatcgattaacatgttgtcgccattga

(19) United States
(12) Patent Application Publication
(10) Pub. No.: US 2015/0176024 A1
(43) Pub. Date: Jun. 25, 2015

(54) TARGET GENES FOR CONTROL OF PLANT PARASITIC NEMATODES AND USE OF SAME

(71) Applicant: NemGenix Pty Ltd, Balcatta WA (AU)

(72) Inventors: John Fosu-Nyarko, Perth (AU); Michael George Kepler Jones, Perth (AU)

(73) Assignee: NEMGENIX PTY LTD, Balcatta WA (AU)

(21) Appl. No.: 14/411,888

(22) PCT Filed: Jun. 28, 2013
(86) PCT No.: PCT/IB2013/001385

§ 371 Date: Dec. 29, 2014

Related U.S. Application Data
(60) Provisional application No. 61/665,799, filed on Jun. 28, 2012.

(51) Int. Cl. C12N 15/82; C12N 15/113

(52) U.S. Cl. CPC C12N 15/8285; C12N 15/8273; C12N 15/1137; C12N 15/113; C12N 2310/14

(57) ABSTRACT

The invention relates to identifying and evaluating target coding and non-coding sequences for control of plant parasitic nematodes by inhibiting one or more biological functions, and their use. The invention provides methods and compositions for identification of such sequences and for the control of a plant-parasitic nematode population. By feeding one or more recombinant double-stranded RNA molecules provided by the invention to the nematode, a reduction in disease may be obtained through suppression of nematode gene expression. The invention is also directed to methods for making transgenic plants that express the double-stranded RNA molecules, and the plant cells and plants obtained thereby.

### CROSS-REFERENCE TO RELATED APPLICATIONS

This application is a national phase entry under 35 U.S.C. §371 of International Patent Application PCT/IB2013/001385, filed Jun. 28, 2013, designating the United States of America and published in English as International Patent Publication WO 2014/001893 A2 on Jan. 3, 2014, which claims the benefit under Article 8 of the Patent Cooperation Treaty and under 35 U.S.C. §119(e) to U.S. Provisional Patent Application Ser. No. 61/665,799, filed Jun. 28, 2012, for “TARGET GENES FOR CONTROL OF PLANT PARASITIC NEMATODES AND USE OF SAME,” the disclosure of each of which is hereby incorporated herein in its entirety by this reference.

### TECHNICAL FIELD

The present invention relates generally to genetic control of plant disease caused by plant-parasitic nematodes. More specifically, the present invention relates to identification of target coding and non-coding sequences, and the use of recombinant DNA technologies for post-transcriptionally repressing or inhibiting expression of target coding and non-coding sequences in the cells of a plant-parasitic nematode to provide a plant protective effect.

### BACKGROUND

Plants are subject to multiple potential disease causing agents, including plant-parasitic nematodes, which are active, flexible, elongate, organisms that live on moist surfaces or in liquid environments, including films of water within soil and moist tissues within other organisms. There are numerous plant-parasitic nematode species, including various cyst nematodes (e.g., Heterodera sp, Globodera sp.), root knot nematodes (e.g., Meloidogyne sp.), root lesion nematodes (e.g., Pratylenchus sp.), dagger nematodes (e.g., Xiphinema sp.) and stem and bulb nematodes (e.g., Ditylenchus sp.), among others. Tylenchid nematodes (members of the order Tylenchida), including the families Heteroderidae, Meloidogynidae, and Pratylenchidae, are the largest and most economically important group of plant-parasitic nematodes. Other important plant-parasitic nematodes include Dorylaimid nematodes (e.g., Xiphinema sp.) among others. Nematode species grow through a series of lifecycle stages and molts.

Typically, there are five stages and four molts: egg stage; J1 (i.e., first juvenile stage); M1 (i.e., first molt); J2 (second juvenile stage; sometimes hatch from egg); M2; J3; M3; J4; M4; A (adult). Juvenile (“J”) stages are also sometimes referred to as larval (“L”) stages. Gene expression may be specific to one or more lifecycle stages.

Some species of nematodes have evolved as very successful parasites of both plants and animals and are responsible for significant economic losses in agriculture and livestock and for morbidity and mortality in humans. Nematode parasites of plants can inhabit all parts of plants, including roots, developing flower buds, leaves, tubers and stems. Plant parasites are classified on the basis of their feeding habits into the broad categories, such as migratory ectoparasites, migratory endoparasites, semi-endoparasites and sedentary endoparasites. Sedentary endoparasites, which include the root knot nematodes (Meloidogyne sp) and cyst nematodes (Globodera sp and Heterodera sp) induce feeding sites (“giant cells” and “syncytia” respectively) and establish long-term infections within roots that are often very damaging to crops. Semi-endoparasites (e.g., Rotylenchulus sp) also induce long term feeding sites, whereas migratory endoparasites (e.g., Pratylenchus sp.) feed from different cells as they move through plant tissues, often causing brown lesions in roots.

Methods and agents for controlling infestations by nematodes have been provided. For example, chemical compositions such as nematocides have typically been applied to soil in which plant parasitic nematodes are present. However, there is a need for safe and effective nematode controls. Chemical agents are often not selective, and exert their effects on non-target organisms, thereby disrupting populations of beneficial microorganisms for a period of time following application of the agent. Chemical agents may persist in the environment and only be slowly metabolized. Nematocidal soil fumigants (e.g., chloropicrin and methyl bromide) are highly toxic, leading to non-renewal of the registration for use of some of these compounds in the United States and other jurisdictions. These agents may also accumulate in the water table or the food chain. These agents may also act as mutagens and/or carcinogens to cause irreversible and deleterious genetic modifications.

Alternative methods for nematode control, such as genetic methods, are increasingly being studied. For example, the organism Caenorhabditis elegans, a bacteriovorous nematode, is the most widely studied nematode genetic model. Public and private databases hold a wealth of information on its genetics and development, but practically applying this information for control of plant-parasitic nematodes remains a challenge. It has previously been impractical to routinely identify a large number of target genes in nematodes other than C. elegans, such as plant-parasitic nematodes, for subsequent functional analysis (e.g., by RNAi analysis). Thus, there has existed a need for improved methods of identifying target genes, suppression of expression of which leads to control of nematode infestation.

Many genes in C. elegans have orthologs in animals, including insects and vertebrates as well as other nematodes. In recent years, a greatly expanded expressed sequence tag (EST) collection has been generated from over 30 parasitic nematode species of plants and animals (Parkinson et al., 2004). For example, thousands of ESTs are available from Heterodera glycines representing portions of approximately 9,000 genes (see, e.g., U.S. patent application Ser. No. 11/360,355, filed Feb. 23, 2006). Conserved genes often retain the same or very similar functions in different nematodes. This functional equivalence has been demonstrated in some cases by transforming C. elegans with homologous genes from other nematodes (Kwa et al., 1995; Redmond et al., 2001). Such equivalence has been shown in cross phyla comparisons for conserved genes and is expected to be more robust among species within a phylum.

RNA interference (RNAi) is a process utilizing endogenous cellular pathways whereby a double-stranded RNA (dsRNA) specific for all or any portion of adequate size of a target gene sequence results in the degradation of the mRNA of interest. In recent years, RNAi has been used to perform gene “knockdown” in a number of species and experimental systems, from the nematode C. elegans, to plants, to insect embryos and cells in tissue culture (Fire et al., 1998; Martinez et al., 2002; McManus and Sharp, 2002). RNAi works through an endogenous pathway including the DICER protein complex that generates about 21-nucleotide small interfering RNAs (siRNAs) from the original dsRNA, often in the form of microRNAs (miRNAs), or from introduced dsRNA, and the RNA-induced silencing complex (RISC) that uses siRNA guides to recognize and degrade the corresponding mRNAs. Only transcripts complementary to the siRNA are cleaved and degraded, and thus the knockdown of mRNA expression is usually sequence specific. In plants, four or more functional groups of DICER genes exist. In Arabidopsis, the DICER genes can generate different sized siRNAs. The gene silencing effect of RNAi persists for days and, under experimental conditions, can lead to a decline in abundance of the targeted transcript of 90% or more, with consequent decline in levels of the corresponding protein. dsRNA-mediated gene suppression by RNAi can be achieved by feeding C. elegans on bacteria expressing double-stranded RNA molecules, by soaking the nematodes in solutions containing double-stranded or small interfering RNA molecules, and by injection of the dsRNA molecules into the nematode. Several large-scale surveys of C. elegans genes by RNAi have been performed such that RNAi knockdown information is available for about 90% of C. elegans genes (see, e.g., Gonczy et al., 2000; Fraser et al., 2000; Sonnichsen et al., 2005).

Currently, only limited published technical or patent information exists on RNAi-mediated gene suppression in plant parasitic nematodes, wherein the double-stranded (dsRNA) or small interfering (siRNA) molecules are taken up from artificial growth media (in vitro) or from plant tissue (in planta). RNAi has been observed to function in several parasitic nematodes including the plant parasites Heterodera glycines and Globodera pallida (Urwin et al., 2002; U.S. Publication US2004/0098761; U.S. Publication US2003/0150017; U.S. Publication US2003/0061626; U.S. Publication US2004/0133943; Fairbairn et al., 2005), Meloidogyne javanica (WO2005/0 19408), and the mammalian parasites Nippostrongylus brasiliensis (Hussein et al., 2002), Brugia malayi (Aboobaker et al., 2003), and Onchocerca volvulus (Lustigman et al., 2004). Production of parasite-specific dsRNA in plant cells has been suggested as a direct strategy for control of plant parasitic nematodes including the soybean cyst nematode, Heterodera glycines (e.g., Fire et al., 1998; U.S. Publication US2004/0098761; WO03/052110 A2; U.S. Publication US2005/0188438). However, no systematic method for identifying target nematode genes for use in such strategies has been reported, and only a limited number of plant-parasitic nematode genes have been proposed as potential targets for RNAi-mediated gene suppression studies.

### BRIEF SUMMARY

The present invention is directed toward compositions and methods for controlling diseases caused by plant-parasitic nematodes. The present invention provides exemplary nucleic acid compositions that are homologous to at least a portion of one or more native nucleic acid sequences in a target plant-parasitic nematode. In certain embodiments, the nematode is selected from Heterodera sp., Meloidogyne sp., Globodera sp., Helicotylenchus sp., Ditylenchus sp., Pratylenchus sp., Paratylenchus sp., Rotylenchus sp., Tylenchulus sp., Tylenchorynchus sp., Hoplolaimus sp., Belonolaimus sp., Anguina sp., Subanguina sp., Nacobbus sp., and Xiphinema sp. In particular embodiments, the nematode may be a Heterodera sp., such as H. glycines. Specific examples of such nucleic acids provided by the invention are included in the attached sequence listing as SEQ ID NOs:1-3, and include:
SEQ ID NO:1-Vacuolar proton-translocating (V-ATPase) subunit E (vha-8)
SEQ ID NO:2-Elongation factor 1 alpha (eft-4)
SEQ ID NO:3-Mismatch Elongation factor 1 alpha (eft-4)

A particular embodiment of the invention provides an isolated polynucleotide selected from the group consisting of: (a) a fragment of at least 19 contiguous nucleotides of a nucleic acid sequence of any of SEQ ID NOs:1-3, as set forth in the sequence listing, wherein contact with or uptake by a plant-parasitic nematode of a double-stranded ribonucleotide sequence comprising at least one strand that is complementary to said fragment inhibits the growth of said nematode; and (b) a complement of the sequence of (a). In another aspect, the invention provides this isolated polynucleotide, further defined as operably linked to a heterologous promoter. In yet another aspect, the invention provides this isolated polynucleotide further defined as comprised on a plant transformation vector. As used herein contact with or uptake by a plant-parasitic nematode includes ingestion of one or more sequences by the nematode, for example, by feeding, by contacting a plant-parasitic nematode with a composition comprising one or more nucleic acid(s) according to the invention, or by soaking of plant-parasitic nematodes with a solution comprising the nucleic acid(s).

In another aspect of the invention, a plant transformation vector may be provided comprising the previously described nucleotide sequence, wherein the sequence contains nucleotide mismatches. These mismatches can be, for example, at about every 20 nucleotides.

Another embodiment includes a plant transformation vector comprising the previously described nucleotide sequence, wherein the sequence is operably linked to a heterologous promoter functional in a plant cell, and to cells transformed with the vector. The cells may be prokaryotic or eukaryotic, and more specifically may be plant cells. Plants and seeds derived from such transformed plant cells are included. Commodity products produced from such a plant, wherein said commodity product comprises a detectable amount of the polynucleotide of the invention or a ribonucleotide expressed therefrom. Methods to produce such a commodity product are also contemplated, by obtaining such transformed plants and preparing food or feed from them. In particular, the food or feed may be oil, meal, protein, starch, sugar, flour, or silage.

Yet another embodiment includes methods for controlling a population of a plant-parasitic nematode, comprising providing an agent having a double-stranded ribonucleotide sequence that functions upon being taken up by the nematode to inhibit a biological function within said nematode, wherein the agent comprises a nucleotide sequence selected from the group consisting of SEQ ID NOs:1-3, and complements thereof. The target sequence may be taken from any part of a gene, including pre-mRNA sequences that may encode a protein, the predicted function of which is selected from the group consisting of: DNA replication, cell cycle control, transcription, RNA processing, translation, ribosome function, tRNA synthesis, tRNA function, protein trafficking, secretion, protein modification, protein stability, protein degradation, energy production, mitochondrial function, intermediary metabolism, cell structure, signal transduction, endocytosis, ion regulation transport, and processes involved in migration of nematodes to plant roots, migration in plant tissues, sensory perception, secretion, parasitism and modification of host plant cells, attraction, motility, nervous system, feeding, digestion, growth, molting, viability, reproduction and embryogenesis.

A particular embodiment provides a method for reducing the number of nematode feeding sites established in the root tissue of a host plant, comprising providing in the host plant a transformed plant cell expressing a polynucleotide sequence of any of SEQ ID NOs:1-3, wherein the polynucleotide is expressed to produce a double-stranded ribonucleic acid that functions upon being taken up by the nematode to inhibit the expression of a target sequence within said nematode and results in a decrease in the number of feeding sites established, or an increase in the ratio of males to females, or a reduction in brood size relative to growth on a host lacking the transformed plant cell.

Another embodiment relates to a method for improving the yield of a crop produced from a crop plant subjected to plant-parasitic nematode infection, the method including: a) introducing a polynucleotide selected from SEQ ID NOs:1-3, into said crop plant; b) cultivating the crop plant to allow the expression of said polynucleotide, wherein expression of the polynucleotide inhibits plant-parasitic nematode infection or growth and loss of yield due to plant-parasitic nematode infection.

An alternative embodiment of the invention provides a method for modulating the expression of a target gene in a plant-parasitic nematode cell, the method comprising: (a) transforming a plant cell with a vector comprising a nucleic acid sequence encoding a dsRNA selected from the group consisting of SEQ ID NOs:1-3, operatively linked to a promoter and a transcription termination sequence; (b) culturing the transformed plant cell under conditions sufficient to allow for development of a plant cell culture including a plurality of transformed plant cells; (c) selecting for transformed plant cells that have integrated the nucleic acid sequence into their genomes; (d) screening the transformed plant cells for expression of the dsRNA encoded by the nucleic acid sequence; and (e) selecting a plant cell that expresses the dsRNA. Plants may also be regenerated from such plant cells.

### BRIEF DESCRIPTION OF THE DRAWINGS

FIG. 1 illustrates an example of T-DNA integrated into Arabidopsis plants according to a particular embodiment of the invention.

FIG. 2 illustrates numbers of cysts per gram dry weight of roots for transgenic events of Vha-8 according to a particular embodiment of the invention.

FIG. 3 illustrates percent reduction in cyst numbers per gram dry weight of roots for transgenic Vha-8 events according to a particular embodiment of the invention.

FIG. 4 illustrates numbers of cysts per gram dry weight of roots for transgenic events of PEf1a events according to a particular embodiment of the invention.

FIG. 5 illustrates percent reduction in cyst numbers per gram dry weight of roots for transgenic PEf1a events according to a particular embodiment of the invention.

FIG. 6 illustrates numbers of cysts per gram dry weight of roots for transgenic events of MEf1a according to a particular embodiment of the invention.

FIG. 7 illustrates percent reduction in cyst numbers per gram dry weight of roots for transgenic MEf1a events according to a particular embodiment of the invention.

### DETAILED DESCRIPTION

The present invention provides methods and compositions for genetic control of plant-parasitic nematode infestations. Methods for identifying genes essential to the lifecycle of a plant-parasitic nematode for use as targets for dsRNA-mediated control of a nematode population are also provided. DNA plasmid vectors encoding dsRNA molecules are designed to suppress nematode genes essential for growth and development. For example, the present invention provides methods and recombinant DNA technologies for post-transcriptionally repressing or inhibiting expression of a target coding or non-coding sequence in a plant-parasitic nematode to provide a protective effect by allowing the plant-parasitic nematode to ingest one or more double-stranded or small interfering ribonucleic acid (siRNA) molecules transcribed from all or a portion of a target sequence, thereby controlling the infection. Thus, the present invention relates to sequence-specific inhibition of expression of coding and non-coding sequences using double-stranded RNA (dsRNA), including small interfering RNA (siRNA) and microRNA (miRNA), to achieve the intended levels of nematode control. A set of isolated and purified nucleotide sequences as set forth in SEQ ID NOs:1-3 is provided. The present invention provides a stabilized dsRNA molecule for the expression of one or more RNAs for inhibition of expression of a target gene in a plant-parasitic nematode, expressed from these sequences and fragments thereof. A stabilized dsRNA, including a dsRNA or siRNA molecule can comprise at least two coding sequences that are arranged in a sense and an antisense orientation relative to at least one promoter, wherein the nucleotide sequence that comprises a sense strand and an antisense strand are linked or connected by a spacer sequence of at least from about five to about one thousand nucleotides, wherein the sense strand and the antisense strand may be a different length, and wherein each of the two coding sequences shares at least 80% sequence identity, at least 90%, at least 95%, at least 98%, or 100% sequence identity, to any one or more nucleotide sequence(s) set forth in SEQ ID NOs:1-3. It is understood that, in some embodiments, such sequence-specific inhibition of expression of coding and non-coding sequences expression from a microRNA can occur without the described defined spacers.

Recombinant DNA constructs include a nucleic acid molecule encoding a dsRNA molecule. The dsRNA may be formed by transcription of one strand of the dsRNA molecule from a nucleotide sequence which is at least from about 80% to about 100% identical to at least a portion of a nucleotide sequence selected from the group consisting of SEQ ID NOs:1-3. Such recombinant DNA constructs may be defined as producing dsRNA molecules capable of inhibiting the expression of endogenous target gene(s) in a plant-parasitic nematode cell upon ingestion. The construct may comprise a nucleotide sequence of the invention operably linked to a promoter sequence that functions in the host cell such as a plant cell. Such a promoter may be tissue-specific and may, for example, be specific to a tissue type which is the subject of plant-parasitic nematode attack. In the case of a root or foliar pathogen, a promoter providing root or leaf-preferred expression, respectively, may be used.

Nucleic acid constructs may include at least one non-naturally occurring nucleotide sequence that can be transcribed into a single-stranded RNA capable of forrning a dsRNA molecule in vivo through intermolecular hybridization. Such dsRNA sequences typically self-assemble and can be provided in the nutrition source of a plant-parasitic nematode to achieve the desired inhibition. A recombinant DNA construct may comprise two or more different non-naturally occurring sequences which, when expressed in vivo as dsRNA sequences and provided in the tissues of the host plant of a plant-parasitic nematode, inhibit the expression of at least two different target genes in the plant-parasitic nematode. In particular embodiments, at least 2, 3, 4, 5, 6, 7, 8, 9 or 10 or more different dsRNAs are produced in a cell, or plant comprising the cell, that have a nematode-inhibitory effect. The dsRNAs may be expressed from multiple constructs introduced in different transformation events or could be introduced on a single nucleic acid molecule, and may also be expressed using a single promoter or multiple promoters. Single dsRNAs may be produced that comprise nucleic acids homologous to multiple loci within one or more plant-parasitic nematodes, both in different populations of the same species of nematode, or from different species of nematodes.

Recombinant DNA constructs may also comprise DNA sequences operatively linked to a plant cell promoter and transcription termination sequence that encode a natural or synthetic miRNA that forms a dsRNA molecule in which the dsRNA portion is at least from about 80% to about 100% identical to at least a portion of a coding or non-coding nucleotide sequence selected from the group consisting of SEQ ID NOs:1-3. The miRNA of such recombinant DNA constructs may be derived either from a native plant miRNA or a native nematode miRNA or a synthetic or modified version thereof.

A recombinant host cell may have in its genome at least one recombinant DNA sequence that is transcribed to produce at least one dsRNA molecule that functions when ingested by a plant-parasitic nematode to inhibit the expression of a target gene in the nematode. The dsRNA molecule may be encoded by any of the aforementioned nucleic acids and as set forth in the sequence listing. A transformed plant cell may have in its genome at least one recombinant DNA sequence described herein. Transgenic plants comprising such a transformed plant cell are also provided, including progeny plants of any generation, seeds, and plant products, each comprising the recombinant DNA. The dsRNA molecules of the present invention may be found in the transgenic plant cell. A sequence selected for use in expression of a gene suppression agent can be constructed from a single sequence derived from one or more target plant-parasitic nematode species and intended for use in expression of an RNA that functions in the suppression of a single gene or gene family in the one or more target pathogens, or that the DNA sequence can be constructed as a chimera from a plurality of DNA sequences.

Fragments of a nucleic acid sequence selected from the group consisting of SEQ ID NOs:1-3 may be defined, for example, as causing the death, growth inhibition, maternal and progeny sterility, reduction in brood size or cessation of infection or feeding by a plant-parasitic nematode, when expressed as a dsRNA and taken up by the nematode. The fragment may, for example, comprise at least about 19, 21, 25, 30, 40, 50, 60, 70, 80, 100 or more contiguous nucleotides of any one or more of the sequences in SEQ ID NOs:1-3, or a complement thereof. Particularly useful will be dsRNA sequences including about 19 to about 300 nucleotides homologous to a nematode target sequence. Also provided is a ribonucleic acid expressed from any of such sequences including a dsRNA.

A method for modulating expression of a target gene in a nematode cell comprises: (a) transforming a plant cell with a vector comprising a nucleic acid sequence encoding a dsRNA operatively linked to a promoter and a transcription termination sequence; (b) culturing the transformed plant cell under conditions sufficient to allow for development of a plant cell culture comprising a plurality of transformed plant cells; (c) selecting for transformed plant cells that have integrated the vector into their genomes; (d) screening the transformed plant cells for expression of the dsRNA encoded by the vector; and (e) selecting a plant cell that expresses the dsRNA. A plant can be regenerated from the plant cell that expresses the dsRNA. Expression of the gene in the plant is sufficient to modulate the expression of a target gene in a cell of a plant-parasitic nematode that contacts the transformed plant or plant cell. Modulation of gene expression may include partial or complete suppression of such expression. In another embodiment, a method for suppression of gene expression in a plant-parasitic nematode comprises providing in the tissue of the host of the nematode a gene-suppressive amount of at least one dsRNA molecule transcribed from a nucleotide sequence as described herein, at least one segment of which is complementary to an mRNA sequence within the cells of the plant-parasitic nematode. A dsRNA molecule, including its modified form such as an siRNA, miRNA, or shRNA molecule, ingested by a pathogenic microorganism in accordance with the invention may be at least from about 80, 81, 82, 83, 84, 85, 86, 87, 88, 89, 90, 91, 92, 93, 94, 95, 96, 97, 98, 99, or about 100% identical to an RNA molecule transcribed from a nucleotide sequence selected from the group consisting of SEQ ID NOs:1-3. Isolated and substantially purified nucleic acid molecules including, but not limited to, non-naturally occurring nucleotide sequences and recombinant DNA constructs for transcribing dsRNA molecules of the present invention are therefore provided, which suppress or inhibit the expression of an endogenous coding sequence or a target coding sequence in the plant-parasitic nematode when introduced thereto.

As used herein, the term “substantially homologous” or “substantial homology,” with reference to a nucleic acid sequence, includes a nucleotide sequence that hybridizes under stringent conditions to the coding sequence of any of SEQ ID NOs:1-3, as set forth in the sequence listing, or the complements thereof. Sequences that hybridize under stringent conditions to any of SEQ ID NOs:1-3, or the complements thereof, are those that allow an antiparallel alignment to take place between the two sequences, and the two sequences are then able, under stringent conditions, to form hydrogen bonds with corresponding bases on the opposite strand to form a duplex molecule that is sufficiently stable under the stringent conditions to be detectable using methods well known in the art. In particular embodiments, substantially homologous sequences have from about 70% to about 80% sequence identity, from about 80% to about 85% sequence identity, from about 85% to about 90% sequence identity, or from about 90% to about 95% sequence identity, to about 99% sequence identity, to the reference nucleotide sequences as set forth in any of SEQ ID NOs:1-3, in the sequence listing, or the complements thereof.

As used herein, the term “ortholog” refers to a gene in two or more species that has evolved from a common ancestral nucleotide sequence, and may retain the same function in the two or more species.

As used herein, the term “sequence identity,” “sequence similarity” or “homology” is used to describe sequence relationships between two or more nucleotide sequences. The percentage of “sequence identity” between two sequences is determined by comparing two optimally aligned sequences over a comparison window, wherein the portion of the sequence in the comparison window may comprise additions or deletions (i.e., gaps) as compared to the reference sequence (which does not comprise additions or deletions) for optimal alignment of the two sequences. The percentage is calculated by determining the number of positions at which the identical nucleic acid base or amino acid residue occurs in both sequences to yield the number of matched positions, dividing the number of matched positions by the total number of positions in the window of comparison, and multiplying the result by 100 to yield the percentage of sequence identity. A sequence that is identical at every position in comparison to a reference sequence is said to be 100% identical to the reference sequence and vice versa. A first nucleotide sequence when observed in the 5′ to 3′ direction is said to be a “complement” of, or complementary to, a second or reference nucleotide sequence observed in the 3′ to 5′ direction if the first nucleotide sequence exhibits complete complementarity with the second or reference sequence.

As used herein, nucleic acid sequence molecules are said to exhibit “complete complementarity” when every nucleotide of one of the sequences read 5′ to 3′ is complementary to every nucleotide of the other sequence when read 3′ to 5′. A nucleotide sequence that is complementary to a reference nucleotide sequence will exhibit a sequence identical to the reverse complement sequence of the reference nucleotide sequence. These terms and descriptions are well defined in the art and are easily understood by those of ordinary skill in the art.

As used herein, the terms “event,” “lines,” and “replicates” are used interchangeably and synonymously.

The term “operably linked,” as used in reference to a regulatory sequence and a structural nucleotide sequence, means that the regulatory sequence causes regulated expression of the linked structural nucleotide sequence. “Regulatory sequences” or “control elements” refer to nucleotide sequences located upstream (5′ noncoding sequences), within, or downstream (3′ non-translated sequences) of a structural nucleotide sequence, and which influence the timing and level or amount of transcription, RNA processing or stability, or translation of the associated structural nucleotide sequence. Regulatory sequences may include promoters, translation leader sequences, introns, enhancers, stem-loop structures, repressor binding sequences, termination sequences, and polyadenylation recognition sequences and the like.

Yield. The term “yield” means a stabilized yield of about 100% or greater relative to the yield of check varieties in the same growing location growing at the same time and under the same conditions. In particular embodiments, “improved yield” or “improving yield” means a cultivar having a stabilized yield of 105% to 115% or greater relative to the yield of check varieties in the same growing location containing significant densities of nematodes that are injurious to that crop growing at the same time and under the same conditions.

As used herein, the terms “drought tolerance” and “osmotic stress tolerance” mean the ability to produce a stabilized or larger crop yield compared to other varieties under drought stress or under reduced plant osmotic conditions brought about by drought conditions. In particular embodiments, “improved yield” or “improving yield” means, in relation to drought tolerance or osmotic stress of a cultivar, having a stabilized yield of 105% to 115% or greater relative to the yield of check varieties in the same growing location containing nematodes that are injurious to that crop growing at the same time and under the same conditions of drought or osmotic stress.

Transgenic plants may contain nucleotide sequences encoding the isolated and substantially purified nucleic acid molecules and the non-naturally occurring recombinant DNA constructs for transcribing the dsRNA molecules for controlling plant-parasitic nematode infections. Such plants may display resistance and/or enhanced tolerance to the infections. Compositions containing the dsRNA nucleotide sequences of the present invention for use in topical applications onto plants or onto animals or into the environment of an animal to achieve the elimination or reduction of plant-parasitic nematode infection are also included.

cDNA sequences encoding proteins or parts of proteins essential for survival, such as amino acid sequences involved in various metabolic or catabolic biochemical pathways, cell division, reproduction, energy metabolism, digestion, parasitism and the like may be selected for use in preparing double-stranded RNA molecules to be provided in the host plant of a plant-parasitic nematode. As described herein, ingestion of compositions by a target organism containing one or more dsRNAs, at least one segment of which corresponds to at least a substantially identical segment of RNA produced in the cells of the target pathogen, can result in the death or other inhibition of the target. A nucleotide sequence, either DNA or RNA, derived from a plant-parasitic nematode can be used to construct plant cells resistant to infestation by the nematode. The host plant of the nematode, for example, can be transformed to contain one or more of the nucleotide sequences derived from the nematode as provided herein. The nucleotide sequence transformed into the host may encode one or more RNAs that form into a dsRNA sequence in the cells or biological fluids within the transformed host, thus making the dsRNA available if/when the plant-parasitic nematode forms a nutritional relationship with the transgenic host. This may result in the suppression of expression of one or more genes in the cells of the plant-parasitic nematode and ultimately death or inhibition of its growth or development. The present invention includes methods for delivery of nematode control agents to plant-parasitic nematodes. Such control agents cause, directly or indirectly, an impairment in the ability of the plant-parasitic nematode to feed, grow or otherwise cause disease in a target host.

The present invention provides in one embodiment a method comprising delivery of stabilized dsRNA molecules to plant-parasitic nematodes as a means for suppression of targeted genes in the plant-parasitic nematode, thus achieving desired control of plant disease in the nematode host. In a particular embodiment, a method of inhibiting expression of a target gene in a plant-parasitic nematode results in the cessation of growth, development, reproduction, and/or feeding, and eventually may result in the death of the plant-parasitic nematode. An embodiment of the method comprises introducing partial or fully stabilized double-stranded RNA (dsRNA) molecules, including its modified forms such as small interfering RNA (siRNA) sequences, into a nutritional composition for the plant-parasitic nematode, and making the nutritional composition or food source available to the plant-parasitic nematode. Ingestion of the nutritional composition containing the double-stranded or siRNA molecules results in the uptake of the molecules by the cells of the nematode, resulting in the inhibition of expression of at least one target gene in the cells of the nematode. Inhibition of the target gene exerts a deleterious effect upon the nematode. The foregoing methods and associated compositions may be used for limiting or eliminating infection or parasitization of a plant or plant cell by a nematode, in or on any host tissue or environment in which the nematode is present by providing one or more compositions comprising the dsRNA molecules described herein in the host of the nematode.

In certain embodiments, dsRNA molecules provided by the invention comprise nucleotide sequences complementary to a sequence, or part thereof, as set forth in any of SEQ ID NOs:1-3, the inhibition of which in a plant-parasitic nematode results in the reduction or removal of a protein or nucleotide sequence agent that is essential for the nematode's growth and development or other biological function. The selected nucleotide sequence may exhibit from about 80% to about 100% sequence identity to one of the nucleotide sequences as set forth in SEQ ID NOs:1-3, including the complement thereof. The sequences identified as having a nematode-protective effect may be readily expressed as dsRNA molecules through the creation of appropriate expression constructs. For example, such sequences can be expressed as a hairpin with stem and loop structure by taking a first segment corresponding to a sequence selected from SEQ ID NOs:1-3, or a fragment thereof, linking this sequence to a second segment spacer region that is not homologous or complementary to the first segment, and linking this to a third segment that transcribes an RNA wherein at least a portion of the third segment is substantially complementary to the first segment. Such a construct forms a stem and loop structure by hybridization of the first segment with the third segment and a loop structure forms comprising the second segment (WO94/01550, WO98/05770, US2002/00488 14 A1, and US2003/001 8993 A1). dsRNA may be generated, for example, in the form of a double-stranded structure such as a stem loop structure (e.g., hairpin), whereby production of siRNA targeted for a nematode sequence is enhanced by co-expression of a fragment of the targeted gene, for instance on an additional plant expressible cassette, that leads to enhanced siRNA production, or reduces methylation to prevent transcriptional gene silencing of the dsRNA hairpin promoter.

The methods and compositions of the present invention may be applied to any monocot and dicot plant, depending on the pathogen (e.g., nematode) control desired. Examples of such transgenic plant cells or transgenic plants of the invention can be obtained by use of any appropriate transient or stable, integrative or non-integrative transformation method known in the art or presently disclosed. The recombinant DNA constructs can be transcribed in any plant cell or tissue or in a whole plant of any developmental stage. Transgenic plants can be derived from any monocot or dicot plant, such as, but not limited to, plants of commercial or agricultural interest, such as crop plants (especially crop plants used for human food or animal feed), wood- or pulp-producing trees, vegetable plants, fruit plants, ornamental plants and “industrial” plants (e.g., sugarcane). Non-limiting examples of plants of interest include grain crop plants (such as wheat, oat, barley, maize, rye, triticale, rice, millet, sorghum, quinoa, amaranth, and buckwheat); forage crop plants (such as forage grasses and forage dicots including alfalfa, vetch, clover, and the like); oilseed crop plants (such as cotton, safflower, sunflower, soybean, canola, rapeseed, flax, peanuts, and oil palm); tree nuts (such as walnut, cashew, hazelnut, pecan, almond, and the like); sugarcane, coconut, date palm, olive, sugarbeet, tea, and coffee; wood- or pulp-producing trees; vegetable crop plants such as legumes (for example, beans, peas, chickpeas, lentils, alfalfa, peanut), lettuce, asparagus, artichoke, celery, carrot, radish, the brassicas (for example, cabbages, kales, mustards, and other leafy brassicas, broccoli, canola, cauliflower, Brussels sprouts, turnip, kohlrabi), edible cucurbits (for example, cucumbers, melons, summer squashes, winter squashes), edible alliums (for example, onions, garlic, leeks, shallots, chives), edible members of the Solanaceae (for example, tomatoes, eggplants, potatoes, peppers, ground cherries), and edible members of the Chenopodiaceae (for example, beets, chard, spinach, quinoa, amaranth); fruit crop plants such as apple, pear, citrus fruits (for example, orange, lime, lemon, grapefruit, and others), stone fruits (for example, apricot, peach, plum, nectarine), banana, pineapple, grape, kiwifruit, papaya, avocado, and berries; and ornamental plants including ornamental flowering plants, ornamental trees and shrubs, ornamental groundcovers, and ornamental grasses. Suitable dicot plants include, but are not limited to, canola, broccoli, cabbage, carrot, cauliflower, Chinese cabbage, cucumber, dry beans, eggplant, fennel, garden beans, gourds, lettuces, melons, okra, peas, peppers, pumpkin, radishes, spinach, squash, watermelon, cotton, potato, quinoa, amaranth, buckwheat, safflower, soybean, sugarbeet, and sunflower. Suitable monocots include, but are not limited to, wheat, oat, barley, maize (including sweet corn and other varieties), rye, triticale, rice, ornamental, forage and amenity grasses, sorghum, millet, onions, leeks, and sugarcane, more preferably maize, wheat, rice and sugarcane.

Exemplary plant-parasitic nematodes from which plants may be protected by the present invention, and their corresponding plants, include, but are not limited to: alfalfa: Ditylenchus dipsaci, Meloidogyne hapla, Meloidogyne incognita, Meloidogyne javanica, Pratylenchus sp., Paratylenchus sp., Xiphinema sp.; banana: Radopholus similis, Helicotylenchus multicinctus, M. incognita, M. arenaria, M. javanica, Pratylenchus coffeae, Rotylenchulus reniformis; beans and peas: Meloidogyne sp., Heterodera sp., Belonolaimus sp., Helicotylenchus sp., Rotylenchulus reniformis, Paratrichodorus anemones, Trichodorus sp.; cassava: Rotylenchulus reniformis, Meloidogyne sp.; beets: Heterodera sp., Nacobbus sp., Pratylenchus sp., Ditylenchus sp., Paratrichodorus sp., Meloidogyne sp.; carrot: Meloidogyne sp, Heterodera sp., Ditylenchus sp. Pratylenchus sp.; cereals: Anguina tritici (Emmer, rye, spelt wheat), Bidera avenae (oat, wheat), Ditylenchus dipsaci (rye, oat), Heterodera avenae, H. filipjevi, H. latipons, H. hordecalis, H. zeae, H. mani, H. bifenestra, H. pakistanensis (cereals and grasses, including wheat, barley, oats, durum wheat, rye, triticale) Subanguina radicicola (oat, barley, wheat, rye), Meloidogyne naasi (barley, wheat, rye), Pratylenchus sp. (oat, wheat, barley, rye), Paratylenchus sp. (wheat), Tylenchorhynchus sp. (wheat, oat); chickpea: Heterodera cajani, Heterodera ciceri, Rotylenchulus reniformis, Hoplolaimus seinhorsti, Meloidogyne artiellia, Meloidogyne sp., Pratylenchus sp., Basirolaimus sp., Bitylenchus sp., Tylenchorhynchyus sp., Hemicriconemoides sp., Xiphinema sp.; citrus: Tylenchulus semipenetrans, Radopholus similis, Radopholus citrophilus, Hemicycliophora arenaria, Pratylenchus sp., Meloidogyne sp., Belonolaimus longicaudatus, Trichodorus sp., Paratrichodorus sp., Xiphinema sp.; clover: Meloidogyne sp., Heterodera trifolii; corn: Pratylenchus sp., Paratrichodorus minor, Longidorus sp., Hoplolairnus columbus, Helicotylenchus sp. Belonolaimus sp., Tylechorhynchus sp., Meloidogyne sp.; cotton: Meloidogyne incognita, Belonolaimus longicaudatus, Rotylenchulus reniformis, Hoplolairnus galeatus, Pratylenchus sp., Tylenchorhynchus sp., Paratrichodorus minor; grapes: Xiphinema sp., Pratylenchus vulnus, Meloidogyne sp., Tylenchulus semipenetrans, Rotylenchulus reniformis; grasses: Pratylenchus sp., Longidorus sp., Paratrichodorus christiei, Xiphinema sp., Ditylenchus sp., Anguina funesta; peanut: Pratylenchus sp., Meloidogyne hapla., Meloidogyne arenaria, Criconemella sp., Belonolaimus longicaudatus; pigeon pea: Heterodera cajani, Rotylenchulus reniformis, Hoplolaimus seinhorsti, Meloidogyne sp., Pratylenchus sp.; potato: Globodera rostochiensis, Globodera pallida, Meloidogyne sp., Pratylenchus sp., Trichodorus primitivus, Ditylenchus sp., Paratrichodorus sp., Nacobbus aberrans; rice: Aphelenchiodes besseyi, Ditylenchus angustus, Hirchmanniella sp., Heterodera oryzae, Meloidogyne sp.; small fruits: Meloidogyne sp.; Pratylenchus sp., Xiphinema sp., Longidorus sp., Paratrichodorus christiei, Aphelenchoides sp.; soybean: Heterodera glycines, Meloidogyne incognita, Meloidogyne javanica, Belonolaimus sp., Pratylenchus sp., Rotylenchulus reniformis, Hoplolaimus columbus; sugar beet: Heterodera schachtii, Ditylenchus dipsaci, Meloidogyne sp., Trichodorus sp., Longidorus sp., Paratrichodorus sp.; sugar cane: Meloidogyne sp., Pratylenchus sp., Radopholus sp., Heterodera sp., Hoplolaimus sp., Helicotylenchus sp., Scutellonema sp., Belonolaimus sp., Tylenchorhynchus sp., Xiphinema sp., Longidorus sp., Paratrichodorus sp.; tobacco: Meloidogyne sp., Pratylenchus sp., Tylenchorhynchus claytoni, Globodera tabacum, Trichodorus sp., Xiphinema americanum, Ditylenchus dipsaci, Paratrichodorus sp.; and tomato: Meloidogyne sp., Pratylenchus sp., Globodera sp., Belonolaimus sp.

Combinations of methods and compositions for controlling infection by plant-parasitic nematodes can also be used. For example, a dsRNA method as described herein for protecting plants from plant-parasitic nematodes includes the additional use of one or more chemical agents or production of protein products that exhibit features different from those exhibited by the dsRNA methods and compositions, and which can interfere with nematode growth or development.

A. Nucleic Acid Compositions and Constructs:

The invention provides recombinant DNA constructs for use in achieving stable transformation of particular host targets. Transformed host targets may express effective levels of preferred dsRNA or siRNA molecules from the recombinant DNA constructs. Pairs of isolated and purified nucleotide sequences may be provided from cDNA library and/or genomic library information. The pairs of nucleotide sequences may be derived from any nematode for use as thermal amplification primers to generate the dsRNA and siRNA molecules of the present invention.

As used herein, the term “nucleic acid” refers to a single- or double-stranded polymer of deoxyribonucleotide or ribonucleotide bases read from the 5′ to the 3′ end. The “nucleic acid” may also optionally contain non-naturally occurring or altered nucleotide bases that permit correct read through by a polymerase and do not reduce expression of a polypeptide encoded by that nucleic acid. The term “nucleotide sequence” or “nucleic acid sequence” refers to both the sense and antisense strands of a nucleic acid as either individual single strands or in the duplex. The term “ribonucleic acid” (RNA) is inclusive of iRNA (inhibitory RNA), dsRNA (double-stranded RNA), siRNA (small interfering RNA including all forms of siRNAs, such as shRNA, tasRNAS, nasiRNA, toRNA and so forth), mRNA (messenger RNA), miRNA (micro-RNA), tRNA (transfer RNA5, whether charged or discharged with a corresponding acylated amino acid), and cRNA (complementary RNA) and the term “deoxyribonucleic acid” (DNA) is inclusive of cDNA and genomic DNA and DNA-RNA hybrids. The terms “nucleic acid segment,” “nucleotide sequence segment,” or more generally “segment” will be understood by those in the art as a functional term that includes both genomic sequences, ribosomal RNA sequences, transfer RNA sequences, messenger RNA sequences, operon sequences and smaller engineered nucleotide sequences that express or may be adapted to express, proteins, polypeptides or peptides.

Provided according to the invention are nucleotide sequences, the expression of which results in an RNA sequence which is substantially homologous to all or part of an RNA molecule of a targeted gene in a plant-parasitic nematode that comprises an RNA sequence encoded by a nucleotide sequence within the genome of the nematode. Thus, after ingestion of the stabilized RNA sequence down-regulation of the nucleotide sequence of the target gene in the cells of the plant-parasitic nematode may be obtained resulting in a deleterious effect on the growth, viability, proliferation, or reproduction of the nematode.

The present invention provides DNA sequences capable of being expressed as an RNA in a cell or microorganism to inhibit target gene expression in a cell, tissue or organ of a plant-parasitic nematode. The sequences comprise a DNA molecule coding for one or more different nucleotide sequences, wherein each of the different nucleotide sequences comprises a sense nucleotide sequence and an antisense nucleotide sequence. The sequences may be connected by a spacer sequence coding for a dsRNA molecule of the present invention. The spacer sequence can constitute part of the sense nucleotide sequence or the antisense nucleotide sequence and forms within the dsRNA molecule between the sense and antisense sequences. The sense nucleotide sequence or the antisense nucleotide sequence is substantially identical to the nucleotide sequence of the target gene or a derivative thereof or a complementary sequence thereto. The dsDNA molecule may be placed operably under the control of a promoter sequence that functions in the cell, tissue or organ of the host expressing the dsDNA to produce dsRNA molecules. In one embodiment, the DNA sequence may be derived from a nucleotide sequence as set forth in SEQ ID NOs:1-3, in the sequence listing.

The invention also provides a DNA sequence for expression in a cell of a plant that, upon expression of the DNA to RNA and ingestion by a plant-parasitic nematode achieves suppression of a target gene in a cell, tissue or organ of a plant-parasitic nematode. The dsRNA may comprise one or multiple structural gene sequences, wherein each of the structural gene sequences comprises a sense nucleotide sequence and an antisense nucleotide sequence that may be connected by a spacer sequence that forms a loop within the complementary and antisense sequences. The sense nucleotide sequence or the antisense nucleotide sequence is substantially identical to the nucleotide sequence of the target gene, derivative thereof, or sequence complementary thereto. The one or more structural gene sequences may be placed operably under the control of one or more promoter sequences, at least one of which is operable in the cell, tissue or organ of a prokaryotic or eukaryotic organism, particularly a plant cell. Methods to express a gene suppression molecule in plants are known (e.g., WO06073727 A2; U.S. Publication US2006/0200878 A1), and may be used to express a nucleotide sequence of the present invention.

A gene sequence or fragment for plant-parasitic nematode control according to the invention may be cloned between two tissue specific promoters, such as two root specific promoters which are operable in a transgenic plant cell and therein expressed to produce mRNA in the transgenic plant cell that form dsRNA molecules thereto. Examples of root specific promoters are known in the art (e.g., the nematode-induced RB7 promoter; U.S. Pat. No. 5,459,252; Opperman et al., 1994). The dsRNA molecules contained in plant tissues are ingested by a plant-parasitic nematode so that the intended suppression of the target gene expression is achieved. By way of example, promoters have been identified that direct gene expression at nematode-induced feeding structures within a plant (e.g., Gheysen and Fenoll, 2002). Thus, a promoter identified from among genes that are reproducibly expressed in feeding sites may be utilized.

A nucleotide sequence provided by the present invention may comprise an inverted repeat separated by a “spacer sequence.” The spacer sequence may be a region comprising any sequence of nucleotides that facilitates secondary structure formation between each repeat, where this is required. In one embodiment of the present invention, the spacer sequence is part of the sense or antisense coding sequence for mRNA. The spacer sequence may alternatively comprise any combination of nucleotides or homologues thereof that are capable of being linked covalently to a nucleic acid molecule.

The nucleic acid molecules or fragments of the nucleic acid molecules or other nucleic acid molecules in the sequence listing are capable of specifically hybridizing to other nucleic acid molecules under certain circumstances. As used herein, two nucleic acid molecules are said to be capable of specifically hybridizing to one another if the two molecules are capable of forming an anti-parallel, double-stranded nucleic acid structure. A nucleic acid molecule is said to be the complement of another nucleic acid molecule if they exhibit complete complementarity. Two molecules are said to be “minimally complementary” if they can hybridize to one another with sufficient stability to permit them to remain annealed to one another under at least conventional “low-stringency” conditions. Similarly, the molecules are said to be complementary if they can hybridize to one another with sufficient stability to permit them to remain annealed to one another under conventional “high-stringency” conditions. Conventional stringency conditions are described by Sambrook, et al., (1989), and by Haynies et al., (1985). Departures from complete complementarity are therefore permissible, as long as such departures do not completely preclude the capacity of the molecules to form a double-stranded structure. Thus, in order for a nucleic acid molecule or a fragment of the nucleic acid molecule to serve as a primer or probe it needs only be sufficiently complementary in sequence to be able to form a stable double-stranded structure under the particular solvent and salt concentrations employed. A nucleic acid for use in the present invention may specifically hybridize to one or more of nucleic acid molecules from a nematode or complements thereof under such conditions. A nucleic acid for use in the present invention may exhibit at least from about 80%, or at least from about 90%, or at least from about 95%, or at least from about 98% or even about 100% sequence identity with one or more nucleic acid molecules as set forth in SEQ ID NOs:1-3, in the sequence listing.

dsRNA or siRNA nucleotide sequences comprise double strands of polymerized ribonucleotide and may include modifications to either the phosphate-sugar backbone or the nucleoside. Modifications in RNA structure may be tailored to allow specific genetic inhibition. In one embodiment, the dsRNA molecules may be modified through an enzymatic process so that siRNA molecules may be generated. The siRNA can efficiently mediate the down-regulation effect for some target genes in some pathogens. This enzymatic process may be accomplished by utilizing an RNAse III enzyme or a DICER enzyme, present in the cells of an insect, a vertebrate animal, a fungus or a plant in the eukaryotic RNAi pathway (Elbashir et al., 2001; Hamilton and Baulcombe, 1999). This process may also utilize a recombinant DICER or RNAse III introduced into the cells of a target nematode through recombinant DNA techniques that are readily known to the skilled in the art. Both the DICER enzyme and RNAse III, being naturally occurring in a pathogen or being made through recombinant DNA techniques, cleave larger dsRNA strands into smaller oligonucleotides. The DICER enzymes specifically cut the dsRNA molecules into siRNA pieces each of which is about 19-25 nucleotides in length. The siRNA molecules produced by either of the enzymes have 2 to 3 nucleotide 3′ overhangs, and 5′ phosphate and 3′ hydroxyl termini. The siRNA molecules generated by RNAse III enzyme are the same as those produced by DICER enzymes in the eukaryotic RNAi pathway and are hence then targeted and degraded by an inherent cellular RNA-degrading mechanism after they are subsequently unwound, separated into single-stranded RNA and hybridize with the RNA sequences transcribed by the target gene. This process results in the effective degradation or removal of the RNA sequence encoded by the nucleotide sequence of the target gene in the pathogen. The outcome is the silencing of a particularly targeted nucleotide sequence within the pathogen. Detailed descriptions of enzymatic processes can be found in Hannon (2002).

B. Recombinant Vectors and Host Cell Transformation:

A recombinant DNA vector may, for example, be a linear or a closed circular plasmid. The vector system may be a single vector or plasmid or two or more vectors or plasmids that together contain the total DNA to be introduced into the genome of the bacterial host. In addition, a bacterial vector may be an expression vector. Nucleic acid molecules as set forth in SEQ ID NOs:1-3, or fragments thereof, can, for example, be suitably inserted into a vector under the control of a suitable promoter that functions in one or more microbial hosts to drive expression of a linked coding sequence or other DNA sequence. Many vectors are available for this purpose, and selection of the appropriate vector will depend mainly on the size of the nucleic acid to be inserted into the vector and the particular host cell to be transformed with the vector. Each vector contains various components depending on its function (amplification of DNA or expression of DNA) and the particular host cell with which it is compatible. The vector components for bacterial transformation generally include, but are not limited to, one or more of the following: a signal sequence, an origin of replication, one or more selectable marker genes, and an inducible promoter allowing the expression of exogenous DNA. Expression and cloning vectors generally contain a selection gene, also referred to as a selectable marker. This gene encodes a protein necessary for the survival or growth of transformed host cells grown in a selective culture medium. Typical selection genes encode proteins that (a) confer resistance to antibiotics or other toxins, e.g., ampicillin, neomycin, methotrexate, or tetracycline, (b) complement auxotrophic deficiencies, or (c) supply critical nutrients not available from complex media, e.g., the gene encoding D-alanine racemase for Bacilli. Those cells that are successfully transformed with a heterologous protein or fragment thereof produce a protein conferring drug resistance and thus survive the selection regimen. An expression vector for producing a mRNA can also contain an inducible promoter that is recognized by a host bacterial organism and is operably linked to the nucleic acid.

The present invention also includes transformation of a nucleotide sequence of the present invention into a plant to achieve nematode-inhibitory levels of expression of one or more dsRNA molecules. A transformation vector can be readily prepared using methods available in the art. The transformation vector comprises one or more nucleotide sequences that is/are capable of being transcribed to an RNA molecule and that is/are substantially homologous and/or complementary to one or more nucleotide sequences encoded by the genome of the target nematode, such that upon uptake of the RNA transcribed from the one or more nucleotide sequences by the target plant-parasitic nematode (or contact between the same), there is down-regulation of expression of at least one of the respective nucleotide sequences of the genome of the nematode. The transformation vector may be termed a dsDNA or RNAi construct and may also be defined as a recombinant molecule, a disease control agent, a genetic molecule or a chimeric genetic construct. A chimeric genetic construct of the present invention may comprise, for example, nucleotide sequences encoding one or more antisense transcripts, one or more sense transcripts, one or more of each of the aforementioned, wherein all or part of a transcript is homologous to all or part of an RNA molecule comprising an RNA sequence encoded by a nucleotide sequence within the genome of a pathogen.

In a particular embodiment, a plant transformation vector comprises an isolated and purified DNA molecule comprising a heterologous promoter operatively linked to one or more nucleotide sequences of the present invention. The nucleotide sequence is selected from the group consisting of SEQ ID NOs:1-3, as set forth in the sequence listing. The nucleotide sequence includes a segment coding all or part of an RNA present within a targeted nematode RNA transcript and may comprise inverted repeats of all or a part of a targeted nematode RNA. The DNA molecule comprising the expression vector may also contain a functional intron sequence positioned either upstream of the coding sequence or even within the coding sequence, and may also contain a five prime (5′) untranslated leader sequence (i.e., a UTR or 5′-UTR) positioned between the promoter and the point of translation initiation. A plant transformation vector may contain sequences from more than one gene, thus allowing production of more than one dsRNA for inhibiting expression of two or more genes in cells of one or more populations or species of target nematodes. A person of skill in the art will readily appreciate that segments of DNA whose sequence corresponds to that present in different genes can be combined into a single composite DNA segment for expression in a transgenic plant. Alternatively, a plasmid of the present invention already containing at least one DNA segment can be modified by the sequential insertion of additional DNA segments between the enhancer and promoter and terminator sequences. In the disease control agent of the present invention designed for the inhibition of multiple genes, the genes to be inhibited can be obtained from the same plant-parasitic nematode species in order to enhance the effectiveness of the control agent. In certain embodiments, the genes can be derived from different plant-parasitic nematodes in order to broaden the range of nematodes against which the agent(s) is/are effective. When multiple genes are targeted for suppression or a combination of expression and suppression, a polycistronic DNA element can be fabricated.

Promoters that function in different plant species are also well known in the art. Promoters useful for expression of polypeptides in plants include those that are inducible, viral, synthetic, or constitutive as described in Odell et al., (1985), and/or promoters that are temporally regulated, spatially regulated, and spatio-temporally regulated. Preferred promoters include the enhanced CaMV35S promoters, and the FMV35S promoter. A fragment of the CaMV35S promoter exhibiting root-specificity may also be preferred. For the purpose of the present invention, it may be preferable to achieve the highest levels of expression of these genes within the root tissues of plants. A number of root-specific promoters have been identified and are known in the art (e.g., U.S. Pat. Nos. 5,110,732; 5,837,848; 5,459,252; Hirel et al., 1992).

A recombinant DNA vector or construct of the present invention may comprise a selectable marker that confers a selectable phenotype on plant cells. Selectable markers may also be used to select for plants or plant cells that contain the exogenous nucleic acids encoding polypeptides or proteins of the present invention. The marker may encode biocide resistance, antibiotic resistance (e.g., kanamycin, Geneticin (G418), bleomycin, hygromycin, etc.), or herbicide resistance (e.g., glyphosate, etc.). Examples of selectable markers include, but are not limited to, a neo gene which codes for kanamycin resistance and can be selected for using kanamycin, G418, etc., a bar gene which codes for bialaphos resistance; a mutant EPSP synthase gene which encodes glyphosate resistance; a nitrilase gene which confers resistance to bromoxynil; a mutant acetolactate synthase gene (ALS) which confers imidazolinone or sulfonylurea resistance; and a methotrexate resistant DHFR gene. Multiple selectable markers are available that confer resistance to ampicillin, bleomycin, chloramphenicol, gentamycin, hygromycin, kanamycin, lincomycin, methotrexate, phosphinothricin, puromycin, spectinomycin, rifampicin, streptomycin and tetracycline, and the like. Examples of such selectable markers are illustrated in U.S. Pat. Nos. 5,550,318; 5,633,435; 5,780,708 and 6,118,047.

A recombinant vector or construct of the present invention may also include a screenable marker. Screenable markers may be used to monitor expression. Exemplary screenable markers include a β-glucuronidase or uidA gene (GUS) which encodes an enzyme for which various chromogenic substrates are known (Jefferson et al., 1987); an R-locus gene, which encodes a product that regulates the production of anthocyanin pigments (red color) in plant tissues (Dellaporta et al., 1988); a β-lactamase gene (Sutcliffe et al., 1978), a gene which encodes an enzyme for which various chromogenic substrates are known (e.g., PADAC, a chromogenic cephalosporin); a luciferase gene (Ow et al., 1986) a xylE gene (Zukowski et al., 1983) which encodes a catechol dioxygenase that can convert chromogenic catechols; an amylase gene (Ikatu et al., 1990); a tyrosinase gene (Katz et al., 1983) which encodes an enzyme capable of oxidizing tyrosine to DOPA and dopaquinone which in turn condenses to melanin; and an α-galactosidase. Preferred plant transformation vectors include those derived from a Ti plasmid of Agrobacterium tumefaciens (e.g., U.S. Pat. Nos. 4,536,475, 4,693,977, 4,886,937, 5,501,967 and EP 0 122 791). Agrobacterium rhizogenes plasmids (or “Ri”) are also useful and known in the art. Other preferred plant transformation vectors include those disclosed, e.g., by Herrera-Estrella (1983); Bevan (1983), Klee (1985) and EP 0 120 516.

Suitable methods for transformation of host cells for use with the current invention include any method by which DNA can be introduced into a cell (see, for example, Mild et al., 1993), such as by transformation of protoplasts (U.S. Pat. No. 5,508,184; Omirulleh et al., 1993), by desiccation/inhibition-mediated DNA uptake (Potrykus et al., 1985), by electroporation (U.S. Pat. No. 5,384,253), by agitation with silicon carbide fibers (Kaeppler et al., 1990; U.S. Pat. No. 5,302,523; and U.S. Pat. No. 5,464,765), by Agrobacterium-mediated transformation (U.S. Pat. Nos. 5,563,055; 5,591,616; 5,693,512; 5,824,877; 5,981,840; 6,384,301) and by acceleration of DNA coated particles (U.S. Pat. Nos. 5,015,580; 5,550,318; 5,538,880; 6,160,208; 6,399,861; 6,403,865; Padgette et al., 945; 1995), etc. Through the application of techniques such as these, the cells of virtually any species may be stably transformed. In the case of multicellular species, the transgenic cells may be regenerated into transgenic organisms. The most widely utilized method for introducing an expression vector into plants is based on the natural transformation system of Agrobacterium. A. tumefaciens and A. rhizogenes are plant pathogenic soil bacteria which genetically transform plant cells. The Ti and Ri plasmids of A. tumefaciens and A. rhizogenes, respectively, carry genes responsible for genetic transformation of the plant. Descriptions of Agrobacterium vector systems and methods for Agrobacterium-mediated gene transfer are provided by numerous references, including Gruber et al., 1993; Mild et al., 1993, Moloney et al., 1989, and U.S. Pat. Nos. 4,940,838 and 5,464,763. Other bacteria such as Sinorhizobium, Rhizobium, and Mesorhizobium that interact with plants naturally can be modified to mediate gene transfer to a number of diverse plants. These plant-associated symbiotic bacteria can be made competent for gene transfer by acquisition of both a disarmed Ti plasmid and a suitable binary vector.

Methods for the creation of transgenic plants and expression of heterologous nucleic acids in plants in particular are known and may be used with the nucleic acids provided herein to prepare transgenic plants that exhibit reduced susceptibility to feeding by a target nematode. Plant transformation vectors can be prepared, for example, by inserting the dsRNA producing nucleic acids disclosed herein into plant transformation vectors and introducing these into plants. One known vector system has been derived by modifying the natural gene transfer system of Agrobacterium tumefaciens. The natural system comprises large Ti (tumor-inducing) plasmids containing a large segment, known as T-DNA, which is transferred to transformed plants. Another segment of the Ti plasmid, the vir region, is responsible for T-DNA transfer. The T-DNA region is bordered by terminal repeats. In the modified binary vectors the tumor-inducing genes have been deleted and the functions of the vir region are utilized to transfer foreign DNA bordered by the T-DNA border sequences. The T-region may also contain a selectable marker for efficient recovery of transgenic plants and cells, and a multiple cloning site for inserting sequences for transfer such as a dsRNA encoding nucleic acid.

A transgenic plant formed using Agrobacterium transformation methods typically contains a single simple recombinant DNA sequence inserted into one chromosome and is referred to as a transgenic event. Such transgenic plants can be referred to as being heterozygous for the inserted exogenous sequence. A transgenic plant homozygous with respect to a transgene can be obtained by sexually mating (selfing) an independent segregant transgenic plant that contains a single exogenous gene sequence to itself, for example, an FO plant, to produce Fl seed. One fourth of the Fl seed produced will be homozygous with respect to the transgene. Germinating Fl seed results in plants that can be tested for heterozygosity, typically using a SNP assay or a thermal amplification assay that allows for the distinction between heterozygotes and homozygotes (i.e., a zygosity assay). Crossing a heterozygous plant with itself or another heterozygous plant results in only heterozygous progeny.

C. Nucleic Acid Expression and Target Gene Suppression:

The present invention includes transformed host plants of a pathogenic target organism, transformed plant cells and transformed plants and their progeny. The transformed plant cells and transformed plants may be engineered to express one or more of the dsRNA or siRNA sequences, under the control of a heterologous promoter, described herein to provide a pathogen-protective effect. These sequences may be used for gene suppression in a pathogen, thereby reducing the level or incidence of disease caused by the pathogen on a protected transformed host organism. As used herein the words “gene suppression” are intended to refer to any of the well-known methods for reducing the levels of protein produced as a result of gene transcription to mRNA and subsequent translation of the mRNA. Gene suppression is also intended to mean the reduction of protein expression from a gene or a coding sequence including post-transcriptional gene suppression and transcriptional suppression. Post-transcriptional gene suppression is mediated by the homology between all or a part of an mRNA transcribed from a gene or coding sequence targeted for suppression and the corresponding double-stranded RNA used for suppression, and refers to the substantial and measurable reduction of the amount of available mRNA available in the cell for binding by ribosomes. The transcribed RNA can be in the sense orientation to effect what is called co-suppression, in the anti-sense orientation to effect what is called anti-sense suppression, or in both orientations producing a dsRNA to effect what is called RNA interference (RNAi).

Transcriptional suppression is mediated by the presence in the cell of a dsRNA gene suppression agent exhibiting substantial sequence identity to a promoter DNA sequence or the complement thereof to effect what is referred to as promoter trans suppression. Gene suppression may be effective against a native plant gene associated with a trait, for example, to provide plants with reduced levels of a protein encoded by the native gene or with enhanced or reduced levels of an affected metabolite. Gene suppression can also be effective against target genes in a plant-parasitic nematode that may ingest or contact plant material containing gene suppression agents, specifically designed to inhibit or suppress the expression of one or more homologous or complementary sequences in the cells of the nematode. Post-transcriptional gene suppression by anti-sense or sense oriented RNA to regulate gene expression in plant cells is disclosed in U.S. Pat. Nos. 5,107,065, 5,759,829, 5,283,184, and 5,231,020.

The use of dsRNA to suppress genes in plants is disclosed in WO 99/53050, WO 99/49029, U.S. Publication No. 2003/0175965, and 2003/0061626, U.S. patent application Ser. No. 10/465,800, and U.S. Pat. Nos. 6,506,559, and 6,326,193. A beneficial method of post transcriptional gene suppression versus a plant-parasitic nematode can employ both sense-oriented and anti-sense-oriented, transcribed RNA which is stabilized, e.g., as a hairpin and stem and loop structure. A DNA construct for effecting post transcriptional gene suppression can be one in which a first segment encodes an RNA exhibiting an anti-sense orientation exhibiting substantial identity to a segment of a gene targeted for suppression, which is linked to a second segment encoding an RNA exhibiting substantial complementarity to the first segment. Such a construct forms a stem and loop structure by hybridization of the first segment with the second segment and a loop structure from the nucleotide sequences linking the two segments (see WO94/01550, WO98/05770, US2002/0048814, and US2003/0018993). Co-expression with an additional target gene segment may also be employed, as noted above (e.g., WO05/01 9408).

In a particular embodiment of the invention, a nucleotide sequence can be used, for which in vitro expression results in transcription of a stabilized RNA sequence that is substantially homologous to an RNA molecule of a targeted gene in a plant-parasitic nematode that comprises an RNA sequence encoded by a nucleotide sequence within the genome of the nematode. Thus, after the plant-parasitic nematode ingests the stabilized RNA sequence, a down-regulation of the nucleotide sequence corresponding to the target gene in the cells of a target nematode is effected.

In certain embodiments of the invention, expression of a fragment of at least 19 contiguous nucleotides of a nucleic acid sequence of any of SEQ ID NOs:1-3 may be utilized, including expression of fragments thereof (e.g., up to 20, 21, 36, 50, 100, or 1000 contiguous nucleotides), or sequences displaying 90-100% identity with such sequences, or their complements. Inhibition of a target gene using the stabilized dsRNA technology of the present invention is sequence-specific in that nucleotide sequences corresponding to the duplex region of the RNA are targeted for genetic inhibition. RNA containing a nucleotide sequence identical to a portion of the target gene is preferred for inhibition. RNA sequences with insertions, deletions, and single-point mutations relative to the target sequence have also been found to be effective for inhibition. In performance of the present invention, it is preferred that the inhibitory dsRNA and the portion of the target gene share at least from about 80% sequence identity, or from about 90% sequence identity, or from about 95% sequence identity, or from about 99% sequence identity, or even about 100% sequence identity. Alternatively, the duplex region of the RNA may be defined functionally as a nucleotide sequence that is capable of hybridizing with a portion of the target gene transcript. A less than full length sequence exhibiting a greater homology compensates for a longer less homologous sequence. The length of the identical nucleotide sequences may be at least about 25, 50, 100, 200, 300, 400, 500 or at least about 1000 bases. Normally, a sequence of greater than 20-100 nucleotides should be used. In particular embodiments, for example, a sequence of greater than about 200-300 nucleotides, and a sequence of greater than about 500-1000 nucleotides may be used, depending on the size of the target gene. The invention has the advantage of being able to tolerate sequence variations that might be expected due to genetic mutation, strain polymorphism, or evolutionary divergence. The introduced nucleic acid molecule may not need to be absolutely homologous, may not need to be full length, relative to either the primary transcription product or fully processed mRNA of the target gene.

In certain embodiments gene expression is inhibited by at least 10%, at least 33%, at least 50%, or, more preferably, by at least 80% within cells in the pathogen, such that a significant inhibition takes place. Significant inhibition is intended to refer to sufficient inhibition that result in a detectable phenotype (e.g., cessation of growth, feeding, development, mortality, etc.) or a detectable decrease in RNA and/or protein corresponding to the target gene being inhibited. Although in certain embodiments of the invention inhibition occurs in substantially all cells of the plant-parasitic nematode, in other preferred embodiments inhibition occurs in only a subset of cells expressing the gene.

dsRNA molecules may be synthesized either in vivo or in vitro. The dsRNA may be formed by a single self-complementary RNA strand or from two complementary RNA strands. Endogenous RNA polymerase of the cell may mediate transcription in vivo or cloned RNA polymerase can be used for transcription in vivo or in vitro. Inhibition may be targeted by specific transcription in an organ, tissue, or cell type; stimulation of an environmental condition (e.g., infection, stress, temperature, chemical inducers); and/or engineering transcription at a developmental stage or age. The RNA strands may or may not be polyadenylated; the RNA strands may or may not be capable of being translated into a polypeptide by a cell's translational apparatus. An RNA, dsRNA, siRNA, or miRNA of the present invention may be produced chemically or enzymatically by one skilled in the art through manual or automated reactions or in vivo in another organism. RNA may also be produced by partial or total organic synthesis; any modified ribonucleotide can be introduced by in vitro enzymatic or organic synthesis. The RNA may be synthesized by a cellular RNA polymerase or a bacteriophage RNA polymerase (e.g., T3, T7, SP6). The use and production of an expression construct are known in the art (see, for example, WO 97/32016; U.S. Pat. Nos. 5,593,874; 5,698,425; 5,712,135; 5,789,214; and 5,804,693). If synthesized chemically or by in vitro enzymatic synthesis, the RNA may be purified prior to introduction into the cell. For example, RNA can be purified from a mixture by extraction with a solvent or resin, precipitation, electrophoresis, chromatography, or a combination thereof. Alternatively, the RNA may be used with no or a minimum of purification to avoid losses due to sample processing. The RNA may be dried for storage or dissolved in an aqueous solution. The solution may contain buffers or salts to promote annealing, and/or stabilization of the duplex strands.

For transcription from a transgene in vivo or an expression construct, a regulatory region (e.g., promoter, enhancer, silencer, and polyadenylation signal) may be used to transcribe the RNA strand (or strands). Therefore, in one embodiment, the nucleotide sequences for use in producing RNA molecules may be operably linked to one or more promoter sequences functional in a microorganism, a fungus, an insect or a plant host cell. The nucleotide sequences may be placed under the control of an endogenous promoter, normally resident in the host genome. The nucleotide sequence of the present invention, under the control of an operably linked promoter sequence, may further be flanked by additional sequences that advantageously affect its transcription and/or the stability of a resulting transcript. Such sequences are generally located upstream of the operably linked promoter and/or downstream of the 3′ end of the expression construct and may occur both upstream of the promoter and downstream of the 3′ end of the expression construct, although such an upstream sequence only is also contemplated.

As used herein, the term “genome” as it applies to cells of a plant-parasitic nematode or a host encompasses not only chromosomal DNA found within the nucleus, but organelle DNA found within subcellular components of the cell. The DNAs of the present invention introduced into plant cells can therefore be either chromosomally integrated or organelle-localized. The term “genome” as it applies to bacteria encompasses both the chromosome and plasmids within a bacterial host cell. The DNAs of the present invention introduced into bacterial host cells can therefore be either chromosomally integrated or plasmid-localized.

As used herein, the term “plant-parasitic nematode” refers to those nematodes that may infect, colonize, parasitize, or cause disease on host plant material transformed to express or coated with a double-stranded gene suppression agent. As used herein, a “nematode resistance” trait is a characteristic of a transgenic plant, transgenic animal, or other transgenic host that causes the host to be resistant to attack from a nematode that typically is capable of inflicting damage or loss to the host. Such resistance can arise from a natural mutation or more typically from incorporation of recombinant DNA that confers plant-parasitic nematode resistance.

To impart nematode resistance to a transgenic plant a recombinant DNA can, for example, be transcribed into an RNA molecule that forms a dsRNA molecule within the tissues or fluids of the recombinant plant. The dsRNA molecule is comprised in part of a segment of RNA that is identical to a corresponding RNA segment encoded from a DNA sequence within a plant-parasitic nematode that prefers to cause disease on the host plant. Expression of the gene within the target plant-parasitic nematode is suppressed by the dsRNA, and the suppression of expression of the gene in the target plant-parasitic nematode results in the plant being resistant to the nematode. Fire et al., U.S. Pat. No. 6,506,599, generically describes inhibition of pest infestation, providing specifics only about several nucleotide sequences that were effective for inhibition of gene function in the nematode species Caenorhabditis elegans. Similarly, US2003/0061626 describes the use of dsRNA for inhibiting gene function in a variety of nematode pests. US2003/0150017 describes using dsDNA sequences to transform host cells to express corresponding dsRNA sequences that are substantially identical to target sequences in specific pests, and particularly describe constructing recombinant plants expressing such dsRNA sequences for ingestion by various plant-parasitic nematodes, facilitating down-regulation of a gene in the genome of the target organism and improving the resistance of the plant to the plant-parasitic nematode. The modulatory effect of dsRNA is applicable to a variety of genes expressed in the plant-parasitic nematode including, for example, endogenous genes responsible for cellular metabolism or cellular transformation, including house-keeping genes, transcription factors, molting-related genes, and other genes which encode polypeptides involved in cellular metabolism or normal growth and development.

As used herein, the phrase “inhibition of gene expression” or “inhibiting expression of a target gene in the cell of a plant-parasitic nematode” refers to the absence (or observable decrease) in the level of protein and/or mRNA product from the target gene. Specificity refers to the ability to inhibit the target gene without manifest effects on other genes of the cell and without any effects on any gene within the cell that is producing the dsRNA molecule. The inhibition of gene expression of the target gene in the plant-parasitic nematode may result in novel phenotypic traits in the nematode.

In another embodiment, the invention provides a delivery system for the delivery of the nematode control agents by ingestion of host cells or the contents of the cells. In accordance with another embodiment, the present invention involves generating a transgenic plant cell or a plant that contains a recombinant DNA construct transcribing the stabilized dsRNA molecules of the present invention. As used herein, “contact with” or “taking up” refers to the process of an agent coming in contact with cells of a target organism, such as a nematode. This may occur, for instance, by nematode feeding, by soaking, or by injection. As used herein, the phrase “generating a transgenic plant cell or a plant” refers to the methods of employing the recombinant DNA technologies readily available in the art (e.g., by Sambrook, et al., 1989) to construct a plant transformation vector transcribing the stabilized dsRNA molecules of the present invention, to transform the plant cell or the plant and to generate the transgenic plant cell or the transgenic plant that contain the transcribed, stabilized dsRNA molecules.

Compositions of the present invention can be incorporated within the seeds of a plant species, either as a product of expression from a recombinant gene incorporated into a genome of the plant cells, or incorporated into a coating or seed treatment that is applied to the seed before planting. The plant cell containing a recombinant gene is considered herein to be a transgenic event. Also included is a delivery system for the delivery of disease control agents to plant-parasitic nematodes. The stabilized dsRNA or siRNA molecules of the present invention may be directly introduced into the cells of a plant-parasitic nematode. Methods for introduction may include direct mixing of RNA with host tissue for the plant-parasitic nematode, as well as engineered approaches in which a species that is a host is engineered to express the dsRNA or siRNA. For example, RNA may be sprayed onto a plant surface. Alternatively, the dsRNA or siRNA may be expressed by microorganisms and the microorganisms may be applied onto a plant surface or introduced into a root, stem by a physical means such as an injection. A plant may also be genetically engineered to express the dsRNA or siRNA in an amount sufficient to kill the plant-parasitic nematodes known to infest the plant. dsRNAs produced by chemical or enzymatic synthesis may also be formulated in a manner consistent with common agricultural practices and used as spray-on products for controlling plant disease. The formulations may include the appropriate stickers and welters required for efficient foliar coverage as well as UV protectants to protect dsRNAs from UV damage. Such additives are commonly used in the bioinsecticide industry and are well known to those skilled in the art. Such applications could be combined with other spray-on insecticide applications, biologically based or not, to enhance plant protection from plant-parasitic nematodes. The present invention also relates to recombinant DNA constructs for expression in a microorganism. Exogenous nucleic acids from which an RNA of interest is transcribed can be introduced into a microbial host cell, such as a bacterial cell or a fungal cell, using methods known in the art.

The nucleotide sequences of the present invention may be introduced into a wide variety of prokaryotic and eukaryotic microorganism hosts to produce the stabilized dsRNA or siRNA molecules. The term “microorganism” includes prokaryotic and eukaryotic species such as bacteria and fungi, as well as nematodes.

D. Transgenic Plants:

Seeds and plants having one or more transgenic event are also included. In one embodiment, a seed having the ability to express a nucleic acid provided herein also has the ability to express at least one other agent, including, but not limited to, an RNA molecule the sequence of which is derived from the sequence of an RNA expressed in a target pathogen such as a nematode and that forms a double-stranded RNA structure upon expressing in the seed or cells of a plant grown from the seed, wherein the ingestion of one or more cells of the plant by the target results in the suppression of expression of the RNA in the cells of the target. In certain embodiments, a seed having the ability to express a dsRNA the sequence of which is derived from a target plant-parasitic nematode also has a transgenic event that provides herbicide tolerance. One beneficial example of a herbicide tolerance gene provides resistance to glyphosate, N-(phosphonomethyl) glycine, including the isopropylamine salt form of such herbicide.

In addition to direct transformation of a plant with a recombinant DNA construct, transgenic plants can be prepared by crossing a first plant having a recombinant DNA construct with a second plant lacking the construct. For example, recombinant DNA for gene suppression can be introduced into first plant line that is amenable to transformation to produce a transgenic plant that can be crossed with a second plant line to introgress the recombinant DNA for gene suppression into the second plant line.

The present invention can be combined with other disease control traits in a plant to achieve desired traits for enhanced control of plant disease. Combining disease control traits that employ distinct modes-of-action can provide protected transgenic plants with superior durability over plants harboring a single control trait because of the reduced probability that resistance will develop in the field.

The invention also includes commodity products containing one or more of the sequences of the present invention, and produced from a recombinant plant or seed containing one or more of the nucleotide sequences of the present invention are specifically contemplated as embodiments of the present invention. A commodity product containing one or more of the sequences of the present invention is intended to include, but not be limited to, meals, oils, sugars, crushed or whole grains or seeds of a plant, or any food product comprising any meal, oil, sugar, or crushed or whole grain of a recombinant plant or seed containing one or more of the sequences of the present invention. The detection of one or more of the sequences of the present invention in one or more commodity or commodity products contemplated herein is de facto evidence that the commodity or commodity product is composed of a transgenic plant designed to express one or more of the nucleotides sequences of the present invention for the purpose of controlling plant disease using dsRNA mediated gene suppression methods.

E. Obtaining Nucleic Acids:

The present invention provides methods for obtaining a nucleic acid comprising a nucleotide sequence for producing a dsRNA or siRNA. One such embodiment includes: (a) analyzing one or more target gene(s) for their expression, function, and phenotype upon dsRNA-mediated gene suppression in a nematode; (b) probing a cDNA or gDNA library with a hybridization probe comprising all or a portion of a nucleotide sequence or a homolog thereof from a targeted nematode that displays an altered, e.g., reduced, nematode growth or development phenotype in a dsRNA-mediated suppression analysis; (c) identifying a DNA clone that hybridizes with the hybridization probe; (d) isolating the DNA clone identified in step (b); (e) sequencing the cDNA or gDNA fragment that comprises the clone isolated in step (d) wherein the sequenced nucleic acid molecule transcribes all or a substantial portion of the RNA sequence or a homolog thereof; and (f) chemically synthesizing all or a substantial portion of a gene sequence, or a siRNA or mRNA or dsRNA corresponding to the present invention.

In another embodiment, a method of the present invention for obtaining a nucleic acid fragment comprising a nucleotide sequence for producing a substantial portion of a dsRNA or siRNA includes: (a) synthesizing first and a second oligonucleotide primers corresponding to a portion of one of the nucleotide sequences from a targeted pathogen; and (b) amplifying a cDNA or gDNA insert present in a cloning vector using the first and second oligonucleotide primers of step (a) wherein the amplified nucleic acid molecule transcribes a substantial portion of a dsRNA or siRNA of the present invention.

In one embodiment, a gene is selected that is essentially involved in the growth, development and reproduction of a plant-parasitic nematode. Other target genes for use in the present invention may include, for example, those that play important roles in nematode viability, movement, migration, growth, development, infectivity, establishment of feeding sites and reproduction. These target genes may be one of the house keeping genes, transcription factors and the like. Additionally, the nucleotide sequences for use in the present invention may also be derived from homologs, including orthologs, of plant, viral, bacterial or insect genes whose functions have been established from literature and the nucleotide sequences of which share substantial similarity with the target genes in the genome of a target nematode. According to one aspect of the present invention for nematode control, the target sequences may essentially be derived from the targeted plant-parasitic nematode. As used herein, the term “derived from” refers to a specified nucleotide sequence that may be obtained from a particular specified source or species, albeit not necessarily directly from that specified source or species. Some of the exemplary target sequences cloned from a nematode that encode proteins or fragments thereof which are homologues of known proteins may be found in the Sequence Listing, for instance SEQ ID NOs:1-3.

For the purpose of the present invention, the dsRNA or siRNA molecules may be obtained by PCR amplification of a target gene sequences derived from a gDNA or cDNA library or portions thereof. The DNA library may be prepared using methods known to those ordinarily skilled in the art and DNA/RNA may be extracted. Genomic DNA or cDNA libraries generated from a target organism may be used for PCR amplification for production of the dsRNA or siRNA. The target genes may be then be PCR amplified and sequenced using the methods readily available in the art. One skilled in the art may be able to modify the PCR conditions to ensure optimal PCR product formation. The confirmed PCR product may be used as a template for in vitro transcription to generate sense and antisense RNA with the included minimal promoters. In one embodiment, the present invention comprises isolated and purified nucleotide sequences that may be used as plant-parasitic nematode control agents. The isolated and purified nucleotide sequences may comprise those as set forth in the sequence listing.

As used herein, the phrase “coding sequence,” “structural nucleotide sequence” or “structural nucleic acid molecule” refers to a nucleotide sequence that is normally translated into a polypeptide, usually via mRNA, when placed under the control of appropriate regulatory sequences. The boundaries of the full length coding sequence are determined by a translation start codon at the 5′-terminus and a translation stop codon at the 3′-terminus. A coding sequence can include, but is not limited to, genomic DNA, cDNA, EST and recombinant nucleotide sequences. The term “recombinant DNA” or “recombinant nucleotide sequence” refers to DNA that contains a genetically engineered modification through manipulation via mutagenesis, restriction enzymes, and the like.

Nucleic acids of the invention can be synthesized by a number of approaches, e.g., Ozaki et al., Nucleic Acids Research, 20:5205-5214 (1992); Agrawal et al., Nucleic Acids Research, 18:5419-5423 (1990); or the like. The nucleic acid of the invention may be conveniently synthesized on an automated DNA synthesizer, e.g., a P.E. Biosystems, Inc. (Foster City, Calif.) model 392 or 394 DNA/RNA Synthesizer, using standard chemistries, such as phosphoramidite chemistry (see, for example, disclosed in the following references: Beaucage et al., Tetrahedron, 48:2223-2311 (1992); Molko et al., U.S. Pat. No. 4,980,460; Koster et al., U.S. Pat. No. 4,725,677; Caruthers et al., U.S. Pat. Nos. 4,415,732; 4,458,066; and 4,973,679). Alternative chemistries resulting in non-natural backbone groups, such as phosphorothioate, phosphoramidate, and the like, can also be employed.

### EXAMPLES

### Example 1

### Identification of Target Genes

To identify target genes for the present invention, a careful study on RNAi phenotypes of genes in Wormbase, the comprehensive database of information on C. elegans, was conducted. C. elegans orthologs identified for the present invention were known to be essential genes for which RNAi disrupted the development, growth and viability of the nematodes at different stages of the life cycle. The functions of these genes can be expected to be conserved in diverse organisms and especially among nematodes. A bioinformatics study of the genes indicated that they were specific enough to avoid off-target effects; more especially their sequences were dissimilar to that of plants, humans and other mammals. Two genes involved in growth and development of the target nematode were analyzed. They are involved in general metabolic processes including protein translation, embryogenesis, body wall contraction, development to adulthood such that their down-regulation in C. elegans affects growth and development, viability and reproduction. The gene sequences, isolated from cyst nematodes, in particular H. schachtii, share close homologies to those of H. glycines.

### Example 2

### Structure and Biology of Target Genes

Vacuolar H⁺-ATPase V1 Sector, Subunit E (vha-8)

The vha-8 gene encodes a vacuolar proton-translocating ATPase required for reproduction in female nematodes, specifically for embryogenesis (embryonic and larval viability), for ovulation, and for proper formation of yolk protein. Expression levels of VHA-8 protein are very low in embryos but increase strongly after hatching and are quite high in the L2 larval stages. The protein is detected in the cytoplasm of excretory canals throughout development, and in the stacked apical plasma membrane sheets of syncytial hypodermal cells. Nematodes defective of Vha-8 are sterile (maternal), grow slowly, do not molt properly, have increased necrotic cells and exhibit no pharyngeal pumping. Other developmental effects include larval arrest and lethality both at the embryonic and larval stages (Simmer et al., 2003; Kamath et al., 2003, Choi et al., 2003. Frand et al., 2005; Ji et al., 2006, Green et al., 2011).

Elongation Factor 1 Alpha (ef1α)

Elongation factor-1 alpha (EF-1α) is an essential component of the eukaryotic translational apparatus. It binds to GTP and catalyzes the binding of aminoacyl-transfer RNAs to ribosomes. It is required for the elongation step of protein synthesis. In mammals, expression of Ef1α decreases towards the end of the lifespan and over-expression prolongs the lifespan of Drosophila melanogaster. In C. elegans several genes are known to encode translation elongation factor homologs. Two of these genes, eft-3 and eft-4, encode Ef1-alpha homologs, known to be required for embryonic viability, fertility, and germline maintenance. Other elongation factor genes in C. elegans are known to be essential for embryonic development, reproduction, and the overall health of the animal. mRNA of the gene is detectable at all stages of C. elegans and is expressed in the pharynx; intestine and body wall muscles of both the larvae and adult stages (Mitrovich & Anderson, 2000; Piano et al., 2000; Kamath et al., 2003).

TABLE 1
RNAi phenotypes of two genes in nematodes (as observed in C. elegans)

| Genes | Selected RNAi phenotypes | References |
|---|---|---|
| Vacuolar | Embryonic lethal, Larval lethal, | Simmer et al., 2003; |
| proton-translocating | Larval arrest, Maternal sterile, Molt | Kamath et al., 2003, |
| (V-ATPase) subunit E | defect, Increased necrotic cell death, | Choi et al., 2003. |
| (vha-8) | Absent pharangeal pumping, | Frand et al., 2005; |
| | Reduced brood size, Slow growth, | Ji et al., 2006; |
| | Sterile progeny, Sterile | Green et al., 2011 |
| Elongation factor 1 alpha | Embryonic lethal, Maternal sterile, | Gonczy et al., 2000; |
| (eft-4) | Larval arrest, Slow growth | Piano et al., 2000; |
| | | Kamath et al., 2003; |
| | | Simmer et al., 2003, |
| | | Rual et al., 2004; |
| | | Sonnichsen et al., 2005 |

### Example 3

### Amplification of Target Genes from H. schachtii

The H. schachtii orthologs of the two genes (SEQ ID NOs:1-3) were obtained using the amino acid sequences derived from C. elegans gene sequences to query several databases including the National Centre for Biotechnology Information (NCBI), Nembase (www.nematode.org) and Nematode.net. Comparative analyses of identified orthologs were then undertaken with those of other parasitic nematodes including plant and animal parasites (such as Brugia malayi) to confirm identity. Available sequences were further analyzed after back-translation using ORFFinder to identify coding regions. In cases where more than one EST was available, contigs were made after multiple alignments to obtain the maximum length of coding region possible. When no EST was available in the database for H. schachtii, that of the closely related H. glycines was used to design primers. These genes were called “perfect gene or sequence or wild type,” as opposed to the modified genes or sequences referred to as “mismatch” in this manuscript. The “mismatch sequence” of Ef1a was made by changing every 20^{th} base of the native sequence (an adenine to thymine and vice versa, and cytosine to guanine and vice versa) without changing the length. It was chemically synthesized and used as template in PCRs in the same way as the “perfect” sequences. ESTs corresponding to the Ef1a gene, of H. glycines and H. schachtii, derived from all stages of the nematode's development were available on public databases (Table 3). After a multiple alignment of H. schachtii ESTs with matches to the C. elegans orthologue (eft-3), primers were designed to amplify a 608 by fragment from H. schachtii cDNA which was then used then to construct a hairpin dsRNA. The cloned sequence was 100% similar to the EST of Hsefia and up to 99% similar to Hgefla (Table 3). A mismatch sequence, MHsef1a, had one in every 20 bases changed, as described previously. Similarly, the vha-8 equivalent was obtained by designing primers based on the ESTs with significant matches to C. elegans orthologues. A 678 by fragment for HsVha-8, was amplified from H. schachtii cDNA and used to generate a hairpin construct targeting the genes. Percent homology of target gene sequences from H. glycines and H. schachtii are shown in Table 2 for available ESTs of each of the two genes.

TABLE 2
Accession numbers of ESTs of H. schachtii and H. glycines target genes

| | Accession numbers of ESTs/sequences from | Percent homology |
|---|---|---|
| | public databases (e.g., Genbank, | between H. glycines |
| | Nematode.net) with highly significant | and H. schachtii |
| Gene | e-values to the cloned H. schachtii target gene | nucleotide sequence |
| Vacuolar | H. glycines | 96-99% |
| proton-translocating | CK349697; CD748301; CB378260; B1748081; | |
| (V-ATPase) subunit | CK350840; CK394468 | |
| E (vha-8) | H. schachtii | |
| | AJ249961.1 | |
| Elongation factor 1 | H. glycines | 98-99% |
| alpha (eft 1-a, eft-3 | CK350770; CK350943; CK350812; BF014023; | |
| and eft-4) | CK348802; CB374291; CB935095; CB380051; | |
| | CB380093; CK351301; B1396585; CK351111; | |
| | CK351574; CK348577 | |
| | H. schachtii | |
| | CF587432; CF100303; CF101683; CF100971; | |
| | CF100079; CF100726; CF101396; CF101356; | |
| | CD750254; CF100571; CF100851 | |

### Example 4

### Primer Design for cDNA of Target Genes

Primers were designed to amplify portions of the coding regions of each target gene sequence: the same primers were used for both perfect and mismatch cDNA without affecting any base changes (Table 3). For directional ligation and cloning of the sense and antisense sequences into the pKannibal hairpin vector, separate primer pairs were designed with restriction endonuclease sites: a pair from Eco RI, Xho I or Kpn I for the sense sequence and two from Cla I, Xba I or Hind III for the antisense sequence (Table 3).

TABLE 3
Primers for amplifying cDNA and
for cloning dsRNA of target genes.

| | Primer for | Primer for amplifying |
|---|---|---|
| Genes | amplifying cDNA | sense and antisense cDNA |
| HsVha-8 | F:- ATGGGCATTTCCGACAACGA | SENSE cDNA PRIMERS |
| | (SEQ ID NO: 4) | F:- TAActcgagATGGGCATTTCCGACAACGA |
| | R:- ATGGGCATTTCCGACAACGA | (SEQ ID NO: 6) |
| | (SEQ ID NO: 5) | R:- TAAggtaccTGATTTTGTCGGCAATGAGG |
| | | (SEQ ID NO: 7) |
| | | ANTISENSE cDNA PRIMERS |
| | | F- TAAtctagaATGGGCATTTCCGACAACGA |
| | | (SEQ ID NO: 8) |
| | | R:- TAAatcgatTGATTTTGTCGGCAATGAGG |
| | | (SEQ ID NO: 9) |
| HsEfla (eft-4) | F:- GCGATTCGCAAGGAAAAGA | SENSE cDNA PRIMERS |
| | (SEQ ID NO: 10) | F- TAACTCGAGGCGATTCGCAAGGAAAA |
| | R:- TAACATGTTGTCGCCATTGA | GA (SEQ ID NO: 12) |
| | (SEQ ID NO: 11) | R- TAAGGTACCTAACATGTTGTCGCCAT |
| | | TGA (SEQ ID NO: 13) |
| | | ANTISENSE cDNA PRIMERS |
| | | F- TAATCTAGAGCGATTCGCAAGGAAA |
| | | AGA (SEQ ID NO: 14) |
| | | R- TAAATCGATTAACATGTTGTCGCCAT |
| | | TGA (SEQ ID NO: 15) |

### Example 5

### Source and Sterilization of H. schachtii

H. schachtii cysts were obtained from soil at a vegetable farm (cabbage, cauliflower and broccoli) at Carabooda, City of Wanneroo, Western Australia. Mature cysts were separated by floatation from soil using a Fenwick Can, the organic matter with cysts was then passed through a coarse sieve (aperture size 850 μm) to remove larger debris and sand grains, and the eluate including cysts, was collected on a second sieve (aperture size 212 μm). The cysts were then transferred using water from a wash bottle onto filter paper supported by a glass filter funnel where they formed a ring around the filter paper at the upper wetted surface. The water was allowed to drain away, and the filter paper plus cysts was dried in an oven at 27° C. Individual cysts were subsequently collected with forceps using a dissecting microscope. Eggs containing second stage juveniles (J2s) were obtained by breaking the cysts open and collecting them by passing through a stack of sieves of aperture sizes 250 μm, 75 μm and 25 μm. The eggs, which collected on the bottom sieve, were then surface sterilized with 3% sodium hypochlorite and or 1% hibitane for 5 minutes followed by 1% streptomycin sulphate and rinsed 5 times with sterile distilled water. J2s were hatched from eggs by soaking the eggs in glass dishes with a solution of 3.14 mM ZnCl₂ at 26-27° C.

### Example 6

### Total RNA Extraction from Eggs and Juveniles of H. schachtii for cDNA Amplification

Total RNA was extracted from a mixture of eggs and second stage juveniles of H. schachtii using TRIZOL® and QIAGEN® RNAEASY® columns. About 3,000 eggs and 1,000 J2s were homogenized in liquid nitrogen in a chilled mortar and pestle previously made RNase-free by treating with 0.1% DEPC, autoclaving at 121° C. and 15 psi, and baking to 150° C. overnight. Half a teaspoonful of the finely ground tissue was quickly added to TRIZOL® (Invitrogen) at 37° C. and mixed well by vortexing. The mixture was incubated on a THERMOMIXER® (Eppendorf) set at room temperature and at 200 rpm for 5 minutes. Two hundred microliters of chloroform was then added to the mixture, which was mixed by vortexing for 15 seconds and incubated at room temperature for a further 1 minute. The contents were vortexed again before centrifuging at 15,000 g on a benchtop Eppendorf centrifuge for 10 minutes to separate phases. The QIAGEN® RNAEASY® column and manufacturer's protocol was then followed to bind and elute RNA from 200 μL of the aqueous phase. Essentially, the sample was transferred to a clean tube containing 700 μL of buffer RLT (previously mixed with 1% β-mercaptoethanol), 500 μL of 96% ethanol was then added, and mixed well by vortexing. The mixture was then transferred to a QIAGEN® MINELUTE® spin column placed in a 2 ml microfuge tube, half at a time, and then spun for 15 seconds at 10,000 g each time. An on-column DNAse I digestion was then performed after which the column with bound RNA was washed and eluted according to the QIAGEN® RNAEASY® Mini Handbook (Ref: Fourth Edition, 2006).

### Example 7

### RT-PCR, Sequencing and Analysis of Clones of Target Genes

Complementary DNA (cDNA) was obtained from total RNA of H. schachtii using a High Capacity cDNA Reverse Transcription Kit (Applied Biosystems, USA) according to the manufacturer's protocol and employing either random hexamers or gene-specific primers. PCRs were prepared with DreamTaq PCR reagents (Quantum Scientific, USA), reactions typically consisting of 1×PCR buffer, 2.0 mM MgCl₂, 0.2 mM dNTPs, 0.5 U of Taq DNA polymerase, 0.5 μM each of primer pair with 30-50 ng of cDNA made to 20 μL with double-distilled deionized water. The PCR reaction was performed in a 0.2 ml microfuge tube or on 96-well PCR plates (Quantum Scientific, USA) in a 2720 Thermal Cycler (Applied Biosystems, USA) or a G-Storm GSI thermal cycler (Gene Technologies Ltd, England). A typical PCR thermal cycling profile was: a single initial denaturation step at 94° C. for 3 minutes, followed by 25-35 cycles of denaturation at 94° C. for 30 seconds, primer annealing at 55° C. for 30 seconds and extension at 72° C. for 1 min. A final incubation step at 72° C. for 7-10 minutes was included to ensure complete extension. PCR products and DNA fragments (after restriction digestion) were cleaned using the Wizard SV Gel and PCR Clean-Up System (Promega Corp, USA) according to the manufacturer's protocol. Purified PCR products were ligated to pGEM-T or pGEM-T Easy cloning vectors in a 1:3-3:1 insert:vector molar ratio. The ligation mix typically consisted of the PCR product in a 1× T4 DNA ligase buffer, 30-50 ng of pGEM-T or pGEM-T Easy with 3 Weiss units of T4 DNA ligase incubated for up to 2 hours at room temperature or 16 hours overnight at 4° C. and the ligated products used to transform E. coli strain JM109 via the heat shock method (Promega Corp., USA). After plasmid preparation using the Wizard Plus Minipreps DNA Purification System (Promega Corp, USA), both strands of DNA were sequenced with capillary electrophoresis using the 3730×1 DNA analyzer (Applied Biosystems) and raw data edited and analyzed with FinchTV (on the World Wide Web at geospiza.com/finchtv/).

### Example 8

### RNAi Constructs and Arabidopsis Transformation

Development of RNAi Constructs:

The pKannibal hairpin vector (Wesley et al., 2001) was used to develop functional RNAi constructs for transforming Arabidopsis. The cDNA of the sense and antisense sequences of the target genes were digested from pGEM-T using appropriate restriction enzymes for directional ligation cloning into the hairpin vectors. Digested DNA (100 ng) of the sense and antisense strands were ligated sequentially to hairpin vectors (75 ng) with T4 DNA Ligase (NEB, USA). A Not I fragment of the RNAi constructs comprising the sense and antisense cDNA of the target gene separated by the pdk intron and placed under the control of the constitutive 35S promoter were subcloned into the binary vector, pART27 to complete a final RNAi construct, each of which was then analyzed by restriction digestion and sequencing before plant transformation.

Description of RNAi Constructs:

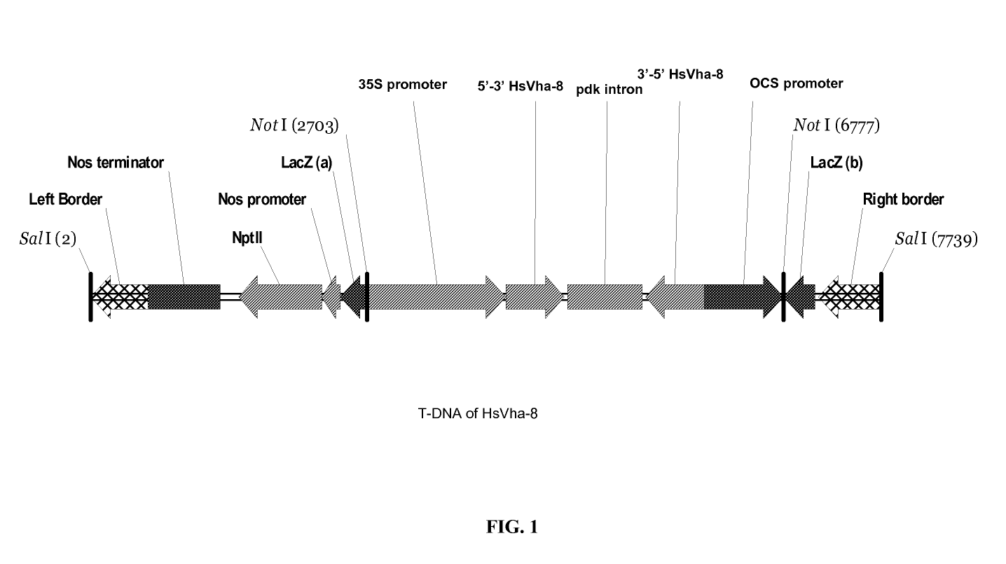

One RNAi construct was made for the vha-8 gene using the native sequences obtained from H. schachtii as a sense and antisense arm of the hairpin (Table 4). Two RNAi constructs were made for the eft-4 gene (Table 4). These were: 1) one RNAi construct using the wild-type sequence as sense and antisense strands in the hairpin and 2) one construct where the mismatch sequence forms the sense and antisense strands in the hairpin; Table 4 shows a number of constructs for each of the target genes and the sequences used in making the arms of the hairpin dsDNA. Each of the hairpin cassettes was digested as a Not I fragment and ligated to pART as in Example 7. An example of the T-DNA integrated into Arabidopsis plants is shown in FIG. 1.

TABLE 4
RNAi constructs for two nematode target genes with
designation of sense and antisense sequences.

| | | Sequence | |
|---|---|---|---|
| | | identity | Sequence identity |
| | | of sense | of antisense |
| Gene | Construct | arm of hairpin | arm of hairpin |
| Vacuolar | pKAPHsVha8 | SEQ ID NO: 1 | SEQ ID NO: 1 |
| proton-translocating | | | |
| (V-ATPase) subunit | | | |
| E (vha-8) | | | |
| Elongation factor 1 | pKAPHsEf1a | SEQ ID NO: 2 | SEQ ID NO: 2 |
| alpha (eft-4) | pKAMHsEf1a | SEQ ID NO: 3 | SEQ ID NO: 3 |

In the representative vector construct, pKAP/MHsG.

p=Conventional designation for plasmid

K=Hairpin vector from which the arms of dsRNA were made, K for pKannibal, with bacterial selective gene, kanamycin.

A=Represents the binary vector carrying the T-DNA, pART27

P=Perfect or native sequence

M=Mismatch sequence

Hs=Heterodera schachtii

G=the nematode gene designed to express as dsRNA in the host plant.

Transformation of Transgenic Plants:

Each of the final RNAi constructs was mobilized into the chemically competent Agrobacterium tumefaciens strain GV3101 Transformations were done using the standard heat shock method, at 37° C. for 5 minutes. In both cases, the mixture was cultured at 28° C. for 3 hours, plated on spectinomycin (50 mg/L) selective plates and kept at 28° C. for 36-48 hours. Arabidopsis thaliana Col 0 growing at 22° C. under 10-14 hours of daylight were transformed with modified Agrobacterium cultures at OD₆₀₀ of 0.8-1.0 using the floral dip method (Clough and Bent, 1998), grown to maturity and the seeds collected.

### Example 9

### Screening and Segregation Analysis of Transgenic Plants

About 1,000 seeds from 15-20 individual T0 lines for each construct were selected for kanamycin resistance. Seeds were surface-sterilized with 100% ethanol for 5 minutes followed by 3% sodium hypochlorite for 20 minutes and then washed 5 times with sterile distilled water. They were then mixed with 0.4% water agar supplemented with 200 mg/L TIMENT1N® and spread on growth media (MS salts with B5 vitamins, 3% sucrose and 0.8% agar) with 50-80 mg/L kanamycin for selection in 10×10 cm sterile plates. The plates were incubated at 22±2° C. under 12 hours of daylight. After 10-15 days, kanamycin-resistant plants at the first true two-leaf stage were transferred to a growth medium with or without selection for up to two weeks to develop roots after which they were transplanted into pots containing seed-raising mix and then grown to produce T1 seeds in a containment glasshouse maintained at 22° C. T1 seeds were then selected as described above to obtain kanamycin-resistant plants capable of expressing the target transgenes.

### Example 10

### Molecular Characterization of Transgenic Plants

Arabidopsis T1 plants are characterized using RT-PCR to amplify mRNA and northern blot analysis to detect siRNA corresponding to transgenes. Small and large scale total RNA isolation for both detection methods were as described by Shi and Bressan (2006). Primers used to detect expression of mRNA of transgenes were designed to bind within the coding regions or can be designed from the intron of the RNAi construct to amplify introns from the pre-processed mRNA. Reverse transcription is done using the High Capacity cDNA Reverse Transcription Kit (Applied Biosystems, USA) and the PCR method described above. Northern blots are done following the protocol described by Wang et al., (2001). Probes for each target gene, used in the Northern analysis, are designed manually, checked for specificity using programs such as Primer 3 (v 0.4.0) and labeled following the manufacturer's protocol.

### Example 11

### Analysis of RNAi

Nematode Infection Assays:

Seeds of T1 Arabidopsis transgenic lines and wild-type Col Ø are surface-sterilized as previously described, selected on kanamycin selective medium for two weeks before transferring them aseptically to culture plates containing Knop's medium at pH 6.4 (Sijmon et al., 1991) solidified with 0.8% Daishin agar (bioWORLD, OH, USA). The plates are sealed with parafilm and kept in a growth room with 12 hour daylight at 26° C. Seedlings are inoculated 10-15 days after germination with 200 J2s per plant. Prior to inoculation, J2s are obtained from surface-sterilized eggs as previously described in Example 5. They are then placed aseptically close to the root tip of plants. Plants are observed 14-28 days after inoculation using a dissecting microscope. White immature or adult female nematodes that develop on each plant root system are counted and used as a measure of nematode susceptibility compared to those of the wild-type and control transgenic plants (containing an empty hairpin cassette with no nematode transgene). The morphology of the plants are also observed visually and compared with that of the wild-type and uninfected plants for any phenotypic differences that may have resulted from nematode infection. Statistical analysis of mean values of nematode females/plant is generated from a target of 10-20 replicates per transgenic line. In a similar nematode infection assay under non-sterile conditions, transgenic Arabidopsis lines with wild controls and wild-type Col Ø are grown in sand or soil in a growth chamber or glasshouse under normal conditions, and then infected with standard numbers of J2s per plant. Infection is subsequently measured 14-28 days or longer after infection, and white immature or adult females or cysts that develop on the plant root systems are counted and used as a measure of nematode susceptibility compared to those of wild-type and control transgenic plants. Root systems can also be treated with a dye (e.g., acid fuchsin) that stains nematodes preferentially, to enhance contrast and count the number of nematodes within or on the surface of infected roots. Statistical analysis of mean values of nematode females/plant is generated from a target of about 10-20 replicates per transgenic line.

Characterization of Nematodes Exposed to Transgenic Plants:

Transcript abundance of nematodes feeding on roots of wild-type and transgenic RNAi plants are analyzed using quantitative/real-time RT-PCR. Depending on the nematode gene knocked out, feeding nematodes that survive are isolated early or later in their development, frozen in liquid nitrogen, and stored for RNA extraction using the modified protocol described in Example 6. Isolated total RNA is then treated with DNase I. Reverse transcription is done with the High-Capacity cDNA Reverse-Transcription Kit and quantitative PCRs are performed in triplicate with Power SYBR Green PCR Master Mix following the manufacturer's protocols (Applied Biosystems, CA, USA). Gene-specific primers for the target genes are designed with Primer 3 (v. 0.4.0). The PCRs are performed as described by Fosu-Nyarko et al., (2009) in a Corbett Rotor Gene RG-3000 (Corbett Research, Brisbane, Queensland, Australia) and relative gene expression analyzed using the 2^{-ΔΔct} method (Livak and Schmittgen 2001). A T-test with one-tailed distribution is then used to determine biological differences in expression between RNAi constructs.

### Example 12

### Transgenic Arabidopsis Lines Expressing Nematode Sequences

Between 10 and 20 transgenic T0 Arabidopsis plants were generated through kanamycin selection and PCR confirmation. A further 10-20 T1 Arabidopsis independent lines expressing hairpin dsRNA for each RNAi construct were obtained for nematode challenge. These were confirmed through kanamycin selection and RT-PCR. Total RNA from selected independent T1 lines is used for RT-PCR with primers designed to bind in the pdk intron of the hairpin cassette in each of the RNAi constructs. In addition, using specific primers for each target gene in an RNAi construct the sequence is amplified to confirm the production of the pre-processed mRNA required for siRNA production in planta. The amplification of the desired bands for each target gene confirms the expression of the short hairpin RNA in each transgenic Arabidopsis. Processing of the dsRNA hairpin of the target genes into siRNA is subsequently confirmed in independent transgenic lines using RNA blot hybridizations.

### Example 13

### Phenotypic Comparison of Transgenic RNAi Lines and Wild-Type Arabidopsis

Target nematode genes or sequences selected for creating hairpin dsRNA had no similarity to any known plant gene or sequence. Hence it was expected that the production or the activation of (systemic) RNAi would not have any deleterious effect on transgenic plants. However, development and morphological characteristics of transgenic lines were compared with wild-type plants as well as those of transgenic lines transformed with an empty hairpin vector. Plant root, shoot, foliage and reproduction characteristics were compared. There were no observable differences in root length and growth patterns of transgenic and wild-type plants. Plant shoot characteristics such as height, leaf numbers and sizes, time of flowering, floral size and appearance were similar. In general, there were no observable morphological differences between transgenic lines and those without expression of target dsRNA when cultured in vitro and in soil in the glasshouse.

### Example 14

### Screening for Resistance to Nematodes in Transgenic Lines

In planta delivery of dsRNA, siRNA or miRNA corresponding to nematode genes and their subsequent uptake by parasitic nematodes through feeding is known to result in down-regulation of the target gene through RNA-mediated gene silencing. When the function of target genes are important, growth, development and reproduction of the said nematode is affected and in the case of plant parasitic nematodes could lead to failure to successfully parasitize, feed, develop and reproduce in the host plant or could lead to death of the nematodes at any stage of development. The choice of target genes and the successful application of RNAi could then be used to control plant parasitic nematodes. Eight to twenty-five replicates of 8-10 independent T1 Arabidopsis transgenic lines were challenged with 1,000 J2s for each RNAi construct. T1 seeds of RNAi lines were germinated on kanamycin selection growth media and resistant plants transferred to growth medium (Example 9) 10 to 15 days after germination. Control seeds, of wild-type Arabidopsis and transgenic lines without a hairpin dsRNA of a nematode gene, were germinated at the same time on growth medium and used for nematode infection. Selected transgenic plants that grew in the presence of kanamycin for two weeks were transferred to a soil-sand potting mix in a growth chamber or glasshouse maintained at 22-23° C., and grown for a further 2-4 weeks to become established. After inoculation with 1,000 J2s, the plants were grown for 3-4 weeks before harvesting and counting the number of nematode cysts that had developed on the roots. This was achieved by gently washing the roots in water, and then observing the roots with a dissecting microscope, to determine how well the nematodes had grown and reproduced in the root system. After counting the number of cysts present the root system of each plant was then dried and weighed to determine the numbers of cysts per gram dry root weight for each plant. From these measurements for each set of replicate plants the average (mean) number of cysts per gram dry weight of root was determined together with the standard error of the mean, and the percentage reduction in cyst numbers compared with control replicates was determined. The ratio of females to males, and development and morphology of surviving nematodes may also be assessed to indicate susceptibility of plants to infection.

### Example 15

### Analysis of RNAi on Nematodes Feeding on Transgenic Lines

For in vitro analysis, feeding nematodes from both control and RNAi lines when present are analyzed to determine the abundance of transcript of each of the target genes from the fourth to the 28^{th} day after infection depending on the target gene. mRNA levels of target genes for nematodes feeding on RNAi Arabidopsis lines are significantly lower than control plants. At the 4^{th}, 7^{th}, 14^{th} and 28^{th} day after infection, transcript levels of target genes in nematode feeding on RNAi lines show a 1.5-2-fold or greater decrease compared to nematodes feeding on control plants (P<0.05). The statistically lower levels of target gene expression in nematodes feeding on RNAi lines is reflected by poor growth and development and the apparent resistance of RNAi lines harboring the dsRNA of the target genes.

### Example 16

### Measurement of Biomass Yield

Nematode infection of susceptible host plants is normally expected to reduce yield losses of the susceptible host in the presence of damaging population densities of plant parasitic nematodes that would normally parasitize that plant. The loss in biomass resulting from nematode infestation varies with the host plant genotype, the nematode species, and the race or genotype of the nematode, and may vary from a few percent to complete plant crop loss. The biomass yield may be measured in replicated pot experiments in a glasshouse or in replicated field tests. In a glasshouse pot experiment, in the order of 8 replicates of both nematode susceptible control (non-transgenic) and transgenic plants of the same genotype expressing coding or non-coding dsRNA to a nematode target gene are grown in soil mix in pots under standard conditions. Once the plant growth is established, J2 juveniles of target nematodes (e.g., cyst-nematodes) are hatched and added to the soil in the pots near the plants in a reproducible manner. Normally, replicated sets of such test plants are challenged with a range of different numbers of nematodes. Depending on the nematode species and the growing conditions, plant biomass is harvested (e.g., 6-8 weeks or longer after nematode inoculation) and weighed (usually wet and dry weights).

Nematode infestation of roots is quantified by standard methods appropriate to the nematode being studied. The average relative weights of biomass of control and transgenic plants can be compared and the effect of nematode infestation on yield can be determined. In the case of a cyst nematode infestation, a reduction in biomass of up to 30% or more may be found relative to transgenic lines expressing recombinant DNA constructs that confer resistance to the nematode used. Similar results may be obtained from replicated field trials, for example, of soybean plants grown in land infested with soybean cyst nematode, in which transgenic plants containing recombinant DNA constructs of specific aspects of the invention should be categorized as moderately resistant to highly resistant in relation to susceptible varieties.

### Example 17

### Bioassay Results for Vha-8 Events

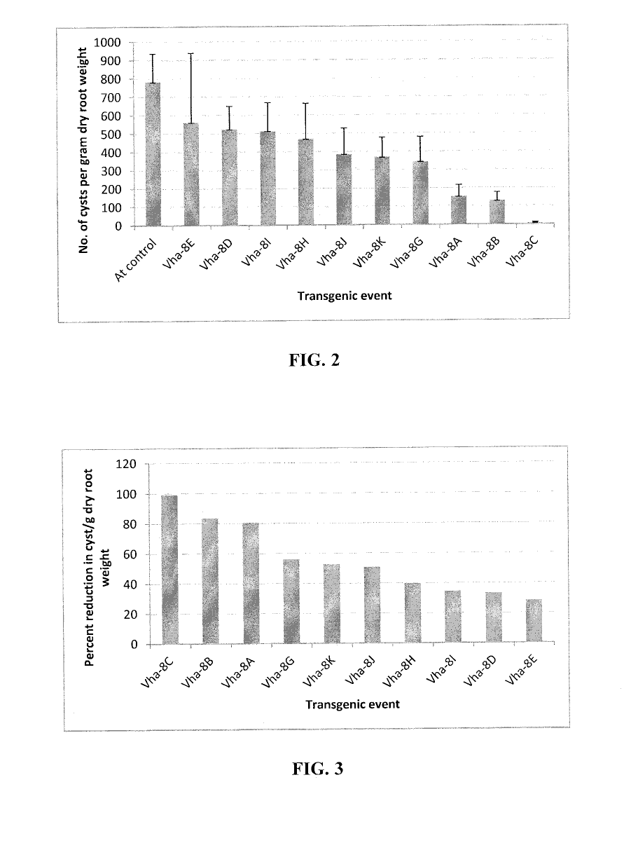

Plants of lines from ten different transgenic events of Arabidopsis thaliana (At) were grown in a sand-soil mixture in a containment glasshouse, infected with J2 BCNs (beet cyst nematodes) (1,000 per plant), and following gentle washing of the roots, the number of cysts that developed on the roots of each plant was counted 3-4 weeks after infection as described in Example 14. The ten different transgenic events contained a construct designed to express dsRNA from sense and antisense segments 100% identical to the BCN Vha-8 gene (PVha-8). The infections were done, with between 11 and 13 lines tested for each transgenic event. The results of the challenges with BCN of different events are provided below (Table 5) as the average number of cysts present per gram dry weight of roots for each event compared to control Arabidopsis plants in the same experiment. The average number of cysts present was obtained from counts of individual lines for each event, and is provided together with standard errors of the mean (FIG. 2, and Table 5). Results are also expressed as the percent reduction in the number of cysts present compared with that present in control plants (FIG. 3). These results clearly show a substantial reduction in the number of cysts compared to non-transgenic control lines, with the percentage reduction in cyst numbers per gram dry weight of roots ranging from 99% for PVha-8C to 28% for PVha-8E.

TABLE 5
Summary of results of BCN challenge experiments for ten
different eventsof transgenic Arabidopsis thaliana plants
expressing dsRNA of BCN gene Vha-8.

| | | | | Percent |
|---|---|---|---|---|
| | | Number | | reduction |
| | | of cysts | | in cyst per |
| Transgenic | No. of lines | per gram dry | Standard | gram dry |
| Event | (n) analyzed | weight of roots | Error | weight of root |
| At control | 11 | 780.7 | 154.6 | |
| Vha-8E | 12 | 560.2 | 378.5 | 28.2 |
| Vha-8D | 13 | 522.3 | 126.7 | 33.1 |
| Vha-8I | 11 | 512.0 | 156.1 | 34.4 |
| Vha-8H | 11 | 469.3 | 194.2 | 39.9 |
| Vha-8J | 13 | 384.6 | 142.7 | 50.7 |
| Vha-8K | 12 | 368.6 | 108.8 | 52.8 |
| Vha-8G | 12 | 343.4 | 136.0 | 56.1 |
| Vha-8A | 13 | 152.4 | 63.5 | 80.5 |
| Vha-8B | 12 | 129.0 | 46.0 | 83.5 |
| Vha-8C | 12 | 7.2 | 5.1 | 99.1 |

### Example 18

### Bioassay Results for Perfect Match and Mismatch Ef1α Events

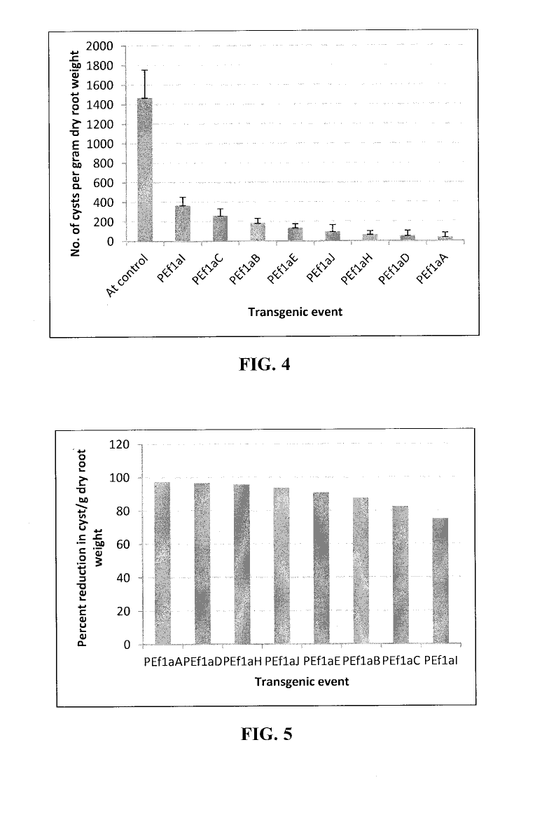

The results of BCN challenge experiments for eight events of PEF1α (perfect dsRNA) transgenic lines are provided below (Table 6). The average number of cysts is provided together with standard errors of the mean (FIG. 4, and Table 6). Results are also expressed as the percent reduction in the number of cysts present compared with that present in control plants (FIG. 5). For PEF1α, the results (cysts per gram dry weight of Arabidopsis roots) showed a very substantial reduction in cyst numbers for different transgenic events, ranging from a 98% reduction for PEf1α-A to 75% reduction for PEf1α-I.

TABLE 6
Summary of results of BCN challenge experiments
for eight different events of transgenic Arabidopsis
thaliana plants expressing dsRNA of BCN gene PEf1a

| | | | | Percent |
|---|---|---|---|---|
| | | Number | | reduction |
| | | of cysts | | in cyst per |
| Transgenic | No. of lines | per gram dry | Standard | gram dry |
| Event | (n) analyzed | weight of root | Error | weight of roots |
| At control | 12 | 1468.3 | 286.2 | |
| PEf1aI | 10 | 366.4 | 86.2 | 75.0 |
| PEf1aC | 19 | 257.5 | 73.5 | 82.5 |
| PEf1aB | 14 | 183.1 | 48.9 | 87.5 |
| PEf1aE | 10 | 133.5 | 41.8 | 90.9 |
| PEf1aJ | 9 | 94.1 | 70.7 | 93.6 |
| PEf1aH | 8 | 64.5 | 37.6 | 95.6 |
| PEf1aD | 13 | 51.3 | 51.3 | 96.5 |
| PEf1aA | 15 | 36.6 | 46.8 | 97.5 |

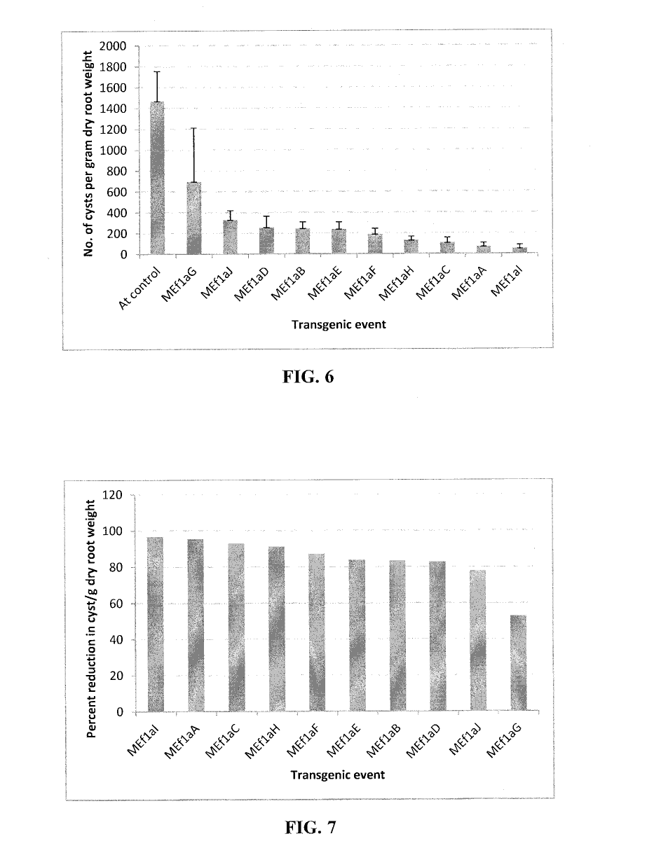

Additionally, the results of BCN challenge experiments for ten events of MEF1α (mismatched dsRNA) transgenic lines are provided below (Table 7). The average number of cysts is provided together with standard errors of the mean (FIG. 6, and Table 7). Results are also expressed as the percent reduction in the number of cysts present compared with that present in control plants (FIG. 7). For MEF1α, the results (cysts per gram dry weight of Arabidopsis roots) showed a very substantial reduction in cyst numbers for different transgenic events, ranging from a 96% reduction for MEf1aI to 52% reduction for MEf1aG.

TABLE 7
Summary of results of two BCN challenge experiments
for six different events of transgenic Arabidopsis thaliana
plants expressing dsRNA of BCN gene MEf1a.

| | | | | Percent |
|---|---|---|---|---|
| | | Number | | reduction |
| | | of cysts | | in cyst per |
| Transgenic | No. of lines | per gram dry | Standard | gram dry |
| Event | (n) analyzed | weight of root | Error | weight of roots |
| At control | 12 | 1468.27 | 286.22 | |
| MEf1aG | 23 | 694.76 | 517.40 | 52.68 |
| MEf1aJ | 19 | 326.43 | 91.43 | 77.77 |
| MEf1aD | 13 | 254.40 | 110.74 | 82.67 |
| MEf1aB | 21 | 244.81 | 63.51 | 83.33 |
| MEf1aE | 20 | 238.30 | 69.59 | 83.77 |
| MEf1aF | 14 | 188.52 | 55.47 | 87.16 |
| MEf1aH | 25 | 129.48 | 37.65 | 91.18 |
| MEf1aC | 7 | 105.10 | 50.43 | 92.84 |
| MEf1aA | 14 | 66.45 | 38.28 | 95.47 |
| MEf1aI | 19 | 48.21 | 37.04 | 96.72 |

In summary, for most lines there were significantly more (>50%) surviving nematodes on controls (wild-type Arabidopsis and transgenic lines without a hairpin dsRNA) than on transgenic Arabidopsis lines harboring the constructs. Whilst all surviving and viable females developed normally on the control plants, development of the few females on the transgenic lines were affected, being smaller in sizes with reduced brood size; with between a reduction of 20% to more than 65% eggs per female. Most tested RNAi lines showed statistically significant reductions in numbers of females (reductions in cysts of up to more than 90%) compared to controls. These results indicate that the transgenic lines showing a reduction in cyst numbers per gram dry weight of root had processed dsRNAs corresponding to nematode target genes and that these were available for uptake by feeding nematodes. More importantly the results indicate RNAi of the two genes affect growth, development and viability of the target nematode. Moreover, RNAi with mismatch sequences with between 90-95% homology to target genes affect nematodes in a similar way to wild-type sequences.

While a number of exemplary aspects and embodiments have been discussed above, those of skill in the art will recognize certain modifications, permutations, additions and sub-combinations thereof. It is therefore intended that the following appended claims and claims hereafter introduced are interpreted to include all such modifications, permutations, additions and sub-combinations as are within their true spirit and scope.

The references discussed herein are provided solely for their disclosure prior to the filing date of the present application. Nothing herein is to be construed as an admission that the inventors are not entitled to antedate such disclosure by virtue of prior invention.

1. An isolated polynucleotide selected from the group consisting of:
(a) a fragment of at least 19 contiguous nucleotides of a nucleic acid sequence of any of: SEQ ID NOs:1-3, wherein contact with a plant-parasitic nematode of a double-stranded ribonucleotide sequence comprising at least one strand that is complementary to said fragment inhibits the growth of said nematode; and
(b) a complement of the sequence of (a).

2. The isolated polynucleotide of claim 1 operably linked to a heterologous promoter.

3. The isolated polynucleotide of claim 1 comprised of a plant transformation vector.

4. A double-stranded ribonucleotide sequence produced from the expression of a polynucleotide according to claim 1, wherein contacting said ribonucleotide sequence with a plant-parasitic nematode inhibits the growth of said nematode.

5. The double-stranded ribonucleotide sequence of claim 4, defined as produced by preparing a recombinant polynucleotide sequence comprising a first, a second and a third polynucleotide sequence, wherein the first polynucleotide sequence comprises the isolated polynucleotide of claim 1, wherein the third polynucleotide sequence is linked to the first polynucleotide sequence by the second polynucleotide sequence, and wherein the third polynucleotide sequence is substantially the reverse complement of the first polynucleotide sequence such that the first and the third polynucleotide sequences hybridize when transcribed into a ribonucleic acid to form the double-stranded ribonucleotide molecule stabilized by the linked second ribonucleotide sequence.

6. The double-stranded ribonucleotide sequence of claim 4, wherein the contacting the polynucleotide sequence with the plant-parasitic nematode inhibits the expression of a nucleotide sequence substantially complementary to said polynucleotide sequence.

7. A plant transformation vector comprising the nucleotide sequence of claim 1, wherein the nucleotide sequence is operably linked to a heterologous promoter functional in a plant cell.

8. A cell transformed with the polynucleotide of claim 1.

9. The cell of claim 8, defined as prokaryotic cell.

10. The cell of claim 8, defined as a eukaryotic cell.

11. The cell of claim 8, defined as a plant cell.

12. A plant transformed with the polynucleotide of claim 1.

13. A seed of the plant of claim 12, wherein the seed comprises the polynucleotide.

14. The plant of claim 12, wherein said polynucleotide is expressed in the plant cell as a double-stranded ribonucleotide sequence.

15. The plant of claim 14, wherein the plant-parasitic nematode is selected from the group consisting of Heterodera sp., Meloidogyne sp., Globodera sp., Helicotylenchus sp., Ditylenchus sp., Pratylenchus sp., Paratylenchus sp., Radopholus sp., Rotylenchus sp., Tylenchulus sp., Tylenchorhynchus sp., Hoplolaimus sp., Belonolaimus sp., Anguina sp., Subanguina sp., Nacobbus sp., and Xiphinema sp.

16. The plant of claim 14, wherein contact between the plant-parasitic nematode and an inhibitory amount of the double-stranded ribonucleotide sequence inhibits growth of the nematode.

17. A commodity product produced from a plant according to claim 12, wherein said commodity product comprises a detectable amount of the polynucleotide of claim 1 or a ribonucleotide expressed therefrom.

18. A method for controlling a plant-parasitic nematode population comprising providing an agent comprising a double-stranded ribonucleotide sequence that functions upon contact with the nematode to inhibit a biological function within said nematode, wherein the agent comprises a nucleotide sequence selected from the group consisting of SEQ ID NOs:1-3, and complements thereof.

19. A method for controlling a plant-parasitic nematode population comprising providing an agent comprising a first polynucleotide sequence that functions upon contact with the pathogen to inhibit a biological function within said nematode, wherein said polynucleotide sequence exhibits from about 90 to about 100% nucleotide sequence identity along at least from about 19 to about 25 contiguous nucleotides to a coding sequence derived from said nematode and is hybridized to a second polynucleotide sequence that is complementary to said first polynucleotide sequence, and wherein said coding sequence derived from said nematode is selected from the group consisting of SEQ ID NOs:1-3, and the complements thereof.

20. The method of claim 19, wherein said nematode is selected from the group consisting of Heterodera sp., Meloidogyne sp., Globodera sp., Helicotylenchus sp., Ditylenchus sp., Pratylenchus sp., Paratylenchus sp., Radopholus sp., Rotylenchus sp., Tylenchulus sp., Tylenchorhynchus sp., Hoplolaimus sp., Belonolaimus sp., Anguina sp., Subanguina sp., Nacobbus sp., and Xiphinema sp.

21. A method for controlling a plant-parasitic nematode population comprising providing in the host plant of a plant-parasitic nematode a transformed plant cell expressing a polynucleotide sequence according to claim 1, wherein the polynucleotide is expressed to produce a double-stranded ribonucleic acid that functions upon contact with the plant-parasitic nematode to inhibit the expression of a target sequence within said nematode and results in decreased growth of the nematode or nematode population, relative to growth on a host lacking the transformed plant cell.

22. The method of claim 21, wherein the nematode exhibits decreased growth following infection of the host plant.

23. The method of claim 21, wherein the target sequence encodes a protein, the predicted function of which is selected from the group consisting of: DNA replication, cell cycle control, transcription, RNA processing, translation, ribosome function, tRNA synthesis, tRNA function, protein trafficking, secretion, protein modification, protein stability, protein degradation, energy production, mitochondrial function, intermediary metabolism, cell structure, signal transduction, endocytosis, ion regulation, transport, and processes involved in migration of external substrate to plant roots, migration of nematodes to plant roots, migration in plant tissues, sensory perception, secretion, parasitism and modification of host plant cells, attraction, motility, nervous system, feeding, digestion, growth, molting, viability, reproduction and embryogenesis.

24. The method of claim 21, wherein said nematode is selected from the group consisting of Heterodera sp., Meloidogyne sp., Globodera sp., Helicotylenchus sp., Ditylenchus sp., Pratylenchus sp., Paratylenchus sp., Radopholus sp., Rotylenchus sp., Tylenchulus sp., Tylenchorhynchus sp., Hoplolaimus sp., Belonolaimus sp., Anguina sp., Subanguina sp., Nacobbus sp., and Xiphinema sp.

25. The method of claim 21, wherein the polynucleotide functions upon contact with the plant-parasitic nematode to suppress a gene that performs a function essential for nematode survival or growth, said function being selected from the group consisting of DNA replication, cell cycle control, transcription, RNA processing, translation, ribosome function, tRNA synthesis, tRNA function, protein trafficking, secretion, protein modification, protein stability, protein degradation, energy production, mitochondrial function, intermediary metabolism, cell structure, signal transduction, endocytosis, ion regulation, transport, and processes involved in migration of external substrate to plant roots, migration in plant tissues, sensory perception, secretion, attraction, motility, nervous system, feeding, digestion, growth, molting, reproduction, embryogenesis.

26. A method of controlling plant nematode pest infestation in a plant comprising providing in the diet of a plant nematode pest a dsRNA comprising:
a) a sense nucleotide sequence; and
b) an antisense nucleotide sequence complementary to said sense nucleotide sequence, wherein said sense nucleotide sequence comprises or is complementary to a nucleotide sequence according to claim 1.

27. The method of claim 26, wherein said diet comprises a plant cell transformed to express said sense and said antisense nucleotide sequence.

28. A method for improving the yield of a crop produced from a crop plant subjected to plant-parasitic nematode infection, said method comprising the steps of:
a) introducing a polynucleotide according to claim 1 into said crop plant;
b) cultivating the crop plant to allow the expression of said polynucleotide;
wherein expression of the polynucleotide inhibits plant-parasitic nematode infection or growth and loss of yield due to plant-parasitic nematode infection.

29. The method of claim 28, wherein the crop plant is selected from the group consisting of maize, wheat, barley, rye, rice, potato, tomato, chickpea, eggplant, cucumber, cabbage, pepper, clover, legume, soybean, pea, alfalfa, clover, sugar cane, sugar beet, silver beet, spinach, tobacco, carrot, cotton, rapeseed (canola), sunflower, safflower, sorghum, strawberry, banana, turf and forage grasses, and fruit and tree crops.

30. The method of claim 28, wherein expression of the polynucleotide produces an RNA molecule that suppresses at least a first target gene in a plant-parasitic nematode that has contacted a portion of said crop plant, wherein the target gene performs at least one essential function selected from the group consisting of DNA replication, cell cycle control, transcription, RNA processing, translation, ribosome function, tRNA synthesis, tRNA function, protein trafficking, secretion, protein modification, protein stability, protein degradation, energy production, mitochondrial function, intermediary metabolism, cell structure, signal transduction, endocytosis, ion regulation, transport, and processes involved in migration of external substrate to plant roots, migration of nematodes to plant roots, migration in plant tissues, sensory perception, secretion, parasitism and modification of host plant cells, attraction, motility, nervous system, feeding, digestion, growth, molting, viability, reproduction and embryogenesis.

31. The method of claim 32, wherein the plant-parasitic nematode is a Tylenchid, Heterodera sp., Heterodera glycines, and all nematode genera and species designated in paragraph 33.

32. A method for improving the osmotic stress tolerance of a crop plant subjected to plant-parasitic nematode infection, said method comprising the steps of:
a) introducing a polynucleotide according to claim 1 into said crop plant;
b) cultivating the crop plant to allow the expression of said polynucleotide; wherein expression of the polynucleotide improves the osmotic stress tolerance of the crop plant.

33. The method of claim 32, wherein the osmotic stress tolerance is defined as drought tolerance.

34. A method of producing a commodity product comprising obtaining a plant according to claim 12 or a part thereof, and preparing a commodity product from the plant or part thereof.

35. A method of producing food or feed, comprising obtaining a plant according to claim 12 or a part thereof and preparing food or feed from said plant or part thereof.

36. The method of claim 35, wherein the food or feed is defined as oil, meal, protein, sugar, starch, flour, silage, biofuels, and plastics.

37. A method for modulating the expression of a target gene in a plant-parasitic nematode cell, the method comprising:
(a) transforming a plant cell with a vector comprising a nucleic acid sequence encoding a dsRNA selected from the group consisting of SEQ ID NOs:1-3, operatively linked to a promoter and a transcription termination sequence;
(b) culturing the transformed plant cell under conditions sufficient to allow for development of a plant cell culture comprising a plurality of transformed plant cells;
(c) selecting for transformed plant cells that have integrated the nucleic acid sequence into their genomes;
(d) screening the transformed plant cells for expression of the dsRNA encoded by the nucleic acid sequence; and
(e) selecting a plant cell that expresses the dsRNA.

38. The method of claim 37, further comprising regenerating a plant from the plant cell that expresses the dsRNA; whereby expression of the gene in the plant is sufficient to modulate the expression of a target gene in a plant-parasitic nematode cell that contacts the transformed plant or plant cell.

39. A method for improving the yield of a crop produced from a crop plant subjected to plant-parasitic nematode infection, said method comprising the steps of:
a) introducing a polynucleotide according to claim 1 into said crop plant;
b) cultivating the crop plant to allow the expression of said polynucleotide; wherein expression of the polynucleotide inhibits plant-parasitic nematode infection, growth, reproduction, or loss of yield due to plant-parasitic nematode infection.

40. The method of claim 35, wherein the crop plant is selected from the group consisting of: maize, wheat, barley, rye, rice, potato, tomato, chickpea, eggplant, cucumber, cabbage, pepper, clover, legume, soybean, pea, alfalfa, clover, sugar cane, sugar beet, silver beet, spinach, tobacco, carrot, cotton, rapeseed (canola), sunflower, safflower, sorghum, strawberry, banana, turf and forage grasses, and fruit and tree crops.

41. The method of claim 39, wherein expression of the polynucleotide produces an RNA molecule that suppresses at least a first target gene in a plant-parasitic nematode that has contacted a portion of said crop plant, wherein the target gene performs at least one essential function selected from the group consisting of DNA replication, cell cycle control, transcription, RNA processing, translation, ribosome function, tRNA synthesis, tRNA function, protein trafficking, secretion, protein modification, protein stability, protein degradation, energy production, mitochondrial function, intermediary metabolism, cell structure, signal transduction, endocytosis, ion regulation, transport, and processes involved in migration of external substrate to plant roots, migration of nematodes to plant roots, migration in plant tissues, sensory perception, secretion, parasitism and modification of host plant cells, attraction, motility, nervous system, feeding, digestion, growth, molting, viability, reproduction and embryogenesis.

42. The method of claim 41, wherein the plant-parasitic nematode is selected from the group consisting of Heterodera sp., Meloidogyne sp., Globodera sp., Helicotylenchus sp., Ditylenchus sp., Pratylenchus sp., Paratylenchus sp., Radopholus sp., Rotylenchus sp., Tylenchulus sp., Tylenchorhynchus sp., Hoplolaimus sp., Belonolaimus sp., Anguina sp., Subanguina sp., Nacobbus sp., and Xiphinema sp.

43. An isolated polynucleotide having greater than about 90% sequence identity to a nucleic acid sequence of any of SEQ ID NOs:1-3.

44. The isolated polynucleotide of claim 43, having greater than about 96% sequence identity to a nucleic acid sequence of any of SEQ ID NOs:1-3.

45. The isolated polynucleotide of claim 43, having greater than about 98% sequence identity to a nucleic acid sequence of any of SEQ ID NOs:1-3.
